(19) United States
(12) Patent Application Publication
(10) Pub. No.: US 2024/0255517 A1
(43) Pub. Date: Aug. 1, 2024

(54) Methods For Screening Particle Formulations Comprising Proteins

(71) Applicant: Elektrofi, Inc., Boston, MA (US)

(72) Inventors: Tyler L. Carter, Newburyport, MA (US); Lyndon Fitzgerald Charles, Jr., Medford, MA (US); Chase Spenser Coffman, Newton, MA (US); Daniel Benjamin Dadon, East Boston, MA (US); Zishu Gui, Boston, MA (US); Sadiqua Shadbar, Allston, MA (US); Chaitanya Sudrik, Stoneham, MA (US); Yi Tang, Medford, MA (US)

(21) Appl. No.: 18/565,193

(22) PCT Filed: Jun. 3, 2022
(86) PCT No.: PCT/US2022/072755

§ 371 Date: Nov. 29, 2023

Related U.S. Application Data
(60) Provisional application No. 63/196,956, filed on Jun. 4, 2021.

(51) Int. Cl. G01N 33/68

(52) U.S. Cl. CPC G01N 33/6803

(57) ABSTRACT

The present disclosure provides screening methods to predict stability of proteins in particle formulations during early stage drug development. In particular, the screening methods disclosed herein uses high throughput protocols that allow rapid identification of stable proteins in the particle formulations in a cost effective manner.

### RELATED APPLICATION(S)

This application claims the benefit of U.S. Provisional Application No. 63/196,956, filed on Jun. 4, 2021. The entire teachings of the above application are incorporated herein by reference.

### GOVERNMENT SUPPORT

This invention was made with government support under Grant No. 1831212 awarded by the National Science Foundation. The government has certain rights in the invention.

### TECHNICAL FIELD

The present disclosure generally relates to the use of screening methods (e.g., high throughput screening methods) to predict stability of proteins in particle formulations, for example, during early stage drug development. In particular, the screening methods disclosed herein can be performed using high throughput protocols to determine the stability of therapeutic biologics in particles, allowing selection of particle formulations in which the biologic has a desired level of stability.

### BACKGROUND

Therapeutic biologics, e.g., therapeutic antibodies (Abs), are large highly complex molecules that are prone to chemical and physical degradations, which often results in loss of biological activity and altered immunogenicity. The selection of optimized particle formulation conditions for therapeutic biologics in early stage drug development has been challenging due to aggressive timelines and reliance on time-consuming extrapolative assays to predict the quality of the particle formulation drug product. High throughput screening (HTS) techniques can accelerate target analysis and allow rapid identification of stable therapeutic biologic particle formulations in a cost effective manner. Moreover, HTS methods can provide large amounts of data to be efficiently collected and used to screen therapeutic biologic conditions for early particle formulation development. Therefore, the development of HTS techniques to identify robust therapeutic biologic particle formulations that can retain physical stability and activity over the drug product shelf life is needed.

### SUMMARY

Provided herein are methods useful for the screening of formulations comprising particles for stability of a protein in the particles. In particular, the screening methods disclosed herein uses high throughput protocols that determine stability of proteins in particles allowing selection of particle formulations.

In one aspect, the disclosure provides a method for screening formulations comprising particles for stability of a protein in the particles, comprising the steps of:
a) dispensing a plurality of different mixtures into a plurality of wells, each mixture comprising a quantity of particles, a first aqueous liquid, and an organic liquid, wherein the particles comprise a protein and one or more pH adjusting agents, and the circularity of the particles is from about 0.80 to about 1.00;
b) removing the first aqueous liquid and the organic liquid from each well to provide a formulation comprising particles in each well;
c) dissolving the particles in a second aqueous liquid and determining the stability of the protein in each well after the first aqueous liquid and the organic liquid have been removed; and
d) selecting a formulation based on the stability of the protein determined in c).

In some embodiments, the mixture comprising a quantity of particles, a first aqueous liquid and an organic liquid in a) is prepared by:
i. adding a quantity of a protein in a first aqueous liquid into containers to provide a plurality of containers, each container comprising a quantity of the protein in the first aqueous liquid;
ii. adding one or more pH adjusting agents to each container;
iii. adding an organic liquid to each container; and
iv. mixing the first aqueous liquid comprising the protein and the pH adjusting agent with the organic liquid in each container, thereby preparing the mixture comprising a quantity of particles, a first aqueous liquid, and an organic liquid in each container, wherein the particles comprise the protein and one or more pH adjusting agents.

The present methods may be useful for the screening of formulations comprising particles for stability of a protein in the particle. In certain embodiments, the screening methods disclosed herein uses high throughput protocols that determine stability of the proteins in the particles allowing selection of particle formulations.

### BRIEF DESCRIPTION OF THE DRAWINGS

The foregoing will be apparent from the following more particular description of example embodiments, as illustrated in the accompanying drawings in which like reference characters, refer to the same parts throughout the different views. The drawings are not necessarily to scale, emphasis instead being placed upon illustrating embodiments.

FIG. 1 shows a general workflow for the HTS method of the disclosure.

FIG. 2 shows an image of circular particles produced using the mixing methods of the disclosure. Scale bar is 30 μm.

FIG. 3 shows a general overview of the analytical workflow for the HTS method of the disclosure.

FIG. 4 shows a graph of the effect of pH and buffering species for BSA.

FIG. 5 shows a graph of excipient selection on the stability of BSA microsphere during storage at 40° C. over 4 weeks (W4) versus TO.

FIG. 6 shows a graph of excipient combinations on the stability of BSA microspheres during storage at 40° C. over 4 weeks (W4) versus TO.

FIG. 7 shows a graph of excipient selection on the stability of BSA microspheres during storage at 40° C. over 4 weeks (W4) versus TO.

FIG. 8 shows a graph of surfactant selection on the stability of BSA microspheres during storage at 40° C. over 4 weeks (W4) versus TO.

FIG. 9 shows a graph of salt selection on the stability of BSA microspheres during storage at 40° C. over 4 weeks (W4) versus TO.

FIG. 10 shows a graph of pH selection on the stability of Rituximab microspheres during storage at 40° C. over 1 week.

FIG. 11 shows a graph of buffering species selection on the stability of Rituximab microspheres during storage at 40° C. over 1 week.

FIG. 12 shows a graph of excipient selection on the stability of Rituximab microspheres during storage at 40° C. over 1 week.

FIGS. 13A-13B show graphs of pH selection on the stability of hIgG microspheres versus Δ (Mon+Dim) % and Δ (Dim+Agg) %.

FIGS. 14A-14B show graphs of buffer selection on the stability of hIgG microspheres versus Δ (Mon+Dim) % and Δ (Dim+Agg) %.

FIGS. 15A-15B show graphs of excipient selection on the stability of hIgG microspheres versus Δ (Mon+Dim) % and Δ (Dim+Agg) %.

FIG. 16 shows an image of particles produced using ultrasonic mixing at 1 uL of feed in 200 uL of n-BA. Scale bar is 10 μm.

FIG. 17 shows an image of particles produced using ultrasonic mixing at 5 uL of feed in 1 mL of n-BA. Scale bar is 10 μm.

FIG. 18 shows a graph comparing the stability of IgG microspheres (“Particles”) to a soluble LDS sample after storage at 40° C. over 12 months.

### DETAILED DESCRIPTION

Considering a typical therapeutic biologic shelf life, e.g., therapeutic monoclonal antibody (mAb) shelf life, developing stabilizing aqueous formulations require significant materials and resources for extended periods of time. The utilization of particle formulation technology can stabilize the therapeutic biologic and extend shelf life by preserving full structure and bioactivity of the therapeutic biologic, e.g., mAb. However, particle formulations of therapeutic biologic drugs, e.g., mAb drugs, can be difficult and time-consuming, mainly due to the complexity of therapeutic biologic structure and the effect of excipients on specific physical and chemical properties of the therapeutic biologic in the solid phase. Thus, understanding protein, e.g., therapeutic biologic, degradation pathways, especially during early stage drug particle development, can be critical for the success of a biopharmaceutical drug particle and a key step in the transformation from a potential drug candidate into a life-saving therapeutic biologic drug.

The present disclosure generally relates to methods for screening formulations comprising particles for stability of a protein in the particles, comprising the steps of: a) dispensing a plurality of different mixtures into a plurality of wells, each mixture comprising a quantity of particles, a first aqueous liquid, and an organic liquid, wherein the particles comprise a protein and one or more pH adjusting agents, and the circularity of the particles is from about 0.80 to about 1.00; b) removing the first aqueous liquid and the organic liquid from each well to provide a formulation comprising particles in each well; c) dissolving the particles in a second aqueous liquid and determining the stability of the protein in each well after the first aqueous liquid and the organic liquid have been removed; and d) selecting a formulation based on the stability of the protein determined in c).

In some embodiments, the mixture comprising a quantity of particles, a first aqueous liquid and an organic liquid in a) is prepared by: i. adding a quantity of a protein in a first aqueous liquid into containers to provide a plurality of containers, each container comprising a quantity of the protein in the first aqueous liquid; ii. adding one or more pH adjusting agents to each container; iii. adding an organic liquid to each container; and iv. mixing the first aqueous liquid comprising the protein and the pH adjusting agent with the organic liquid in each container, thereby preparing the mixture comprising a quantity of particles, a first aqueous liquid, and an organic liquid in each container, wherein the particles comprise the protein and one or more pH adjusting agents. See Example 1 herein for description of an example method of the disclosure for screening formulations comprising particles for stability of a protein in the particles.

It will be readily understood that the aspects and embodiments, as generally described herein, are exemplary. The following more detailed description of various aspects and embodiments are not intended to limit the scope of the present disclosure, but is merely representative of various aspects and embodiments. Moreover, the methods disclosed herein may be changed by those skilled in the art without departing from the scope of the present disclosure. Unless defined otherwise, all technical and scientific terms used herein have the same meaning as is commonly understood by one of skill in the art to which this disclosure belongs. All publications and patents referred to herein are incorporated by reference.

### Definitions

For purposes of the present disclosure, the following definitions will be used unless expressly stated otherwise:

The terms “a”, “an”, “the” and similar referents used in the context of describing the present disclosure are to be construed to cover both the singular and the plural, unless otherwise indicated herein or clearly contradicted by context. All methods described herein, can be performed in any suitable order unless otherwise indicated herein or otherwise clearly contradicted by context. The use of any and all examples, or exemplary language (e.g., “such as”) provided herein, is intended merely to better illuminate the present disclosure and does not pose a limitation on the scope of the disclosure otherwise claimed. No language in the present specification should be construed as indicating any unclaimed element is essential to the practice of the disclosure.

The term “about” in relation to a given numerical value, such as for temperature and period of time, is meant to include numerical values within 10% of the specified value.

As used herein, an “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, iso-butyl, sec-butyl, tert-butyl, n-pentyl, tert-pentyl, neo-pentyl, iso-pentyl, sec-pentyl, 3-pentyl, sec-iso-pentyl, active-pentyl, hexyl, heptyl, octyl, ethylhexyl, and the like. A C₁₋₈ straight chained or branched alkyl group is also referred to as a “lower alkyl” group. An alkyl group with two open valences is sometimes referred to as an alkylene group, such as methylene, ethylene, propylene and the like. Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, an alkyl, a halogen, a hydroxyl, a carbonyl (such as a carboxyl, and alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamide, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN and the like. In some embodiments, the term “alkyl” can mean “cycloalkyl” which refers to a non-aromatic carbocyclic ring having 3 to 10 carbon ring atoms, which are carbon atoms bound together to form the ring. The ring may be saturated or have one or more carbon-carbon double bonds. Examples of cycloalkyl include, but not limited to cyclopropyl, cyclobutyl, cyclopentyl, cyclopentenyl, cyclohexyl, cyclohexenyl, and cycloheptyl, as well as bridged and caged saturated ring groups such as norbornyl and adamantyl. As described herein, organic solvents include, but are not limited to aliphatic hydrocarbon solvents, aromatic hydrocarbon solvents, alcohols or alkylalcohols, alkylethers, sulfoxides, alkylketones, alkylacetates, trialkylamines, alkylformates, trialkylamines, or a combination thereof. Aliphatic hydrocarbon solvents can be pentane, hexane, heptane, octane, cyclohexane, and the like or a combination thereof. Aromatic hydrocarbon solvents can be benzene, toluene, and the like or a combination thereof. Alcohols or alkylalcohols include, for example, methanol, ethanol, propanol, butanol, pentanol, hexanol, heptanol, octanol, decanol, amylalcohol, or a combination thereof. Alkylethers include methyl, ethyl, propyl, butyl, and the like, e.g., diethylether, diisopropylether or a combination thereof. Sulfoxides include dimethyl sulfoxide (DMSO), decylmethyl sulfoxide, tetradecylmethyl sulfoxide, and the like or a combination thereof. The term “alkylketone” refers to a ketone substituted with an alkyl group, e.g., acetone, ethylmethylketone, and the like or a combination thereof. The term “alkylacetate” refers to an acetate substituted with an alkyl group, e.g., ethylacetate, propylacetate (n-propylacetate, iso-propylacetate), butylacetate (n-butylacetate, iso-butylacetate, sec-butylacetate, tert-butylacetate), amylacetate (n-pentylacetate, tert-pentylacetate, neo-pentylacetate, iso-pentylacetate, sec-pentylacetate, 3-pentylacetate, sec-iso-pentylacetate, active-pentylacetate), 2-ethylhexylacetate, and the like or a combination thereof. The term “alkylformate” refers to a formate substituted with an alkyl group, e.g., methylformate, ethylformate, propylformate, butylformate, and the like or a combination thereof. The term “trialkylamine” refers to an amino group substituted with three alkyl groups, e.g., triethylamine.

As used herein, an “amino acid” or “residue” refers to any naturally or non-naturally occurring amino acid, any amino acid derivative or any amino acid mimic known in the art. Included are the L- as well as the D-forms of the respective amino acids, although the L-forms are usually preferred. In some embodiments, the term relates to any one of the 20 naturally occurring amino acids: glycine (Gly), alanine (Ala), valine (Val), leucine (Leu), isoleucine (Ile), proline (Pro), cysteine (Cys), methionine (Met), serine (Ser), threonine (Thr), glutamine (Gin), asparagine (Asn), glutamic acid (Glu), aspartic acid (Asp), lysine (Lys), histidine (His), arginine (Arg), phenylalanine (Phe), tryptophan (Trp), and tyrosine (Tyr) in their L-form. In some embodiments, the amino acid side-chain may be a side-chain of Gly, Ala, Val, Leu, Ile, Met, Cys, Ser, Thr, Trp, Phe, Lys, Arg, His, Tyr, Asn, Gln, Asp, Glu, or Pro. In certain embodiments, the amino acid derivative is an amino acid salt, e.g., amino acid salts of hydrochloric acid, phosphoric acid, DL-lactic/glycolic acids, succinic acid, citric acid, sulfuric acid, sodium hydroxide, potassium hydroxide, sodium succinate, sodium phosphate, sodium acetate, sodium citrate, sodium sulfate, or the like.

As used herein, except where the context requires otherwise, the term “comprise” and variations of the term, such as “comprising”, “comprises” and “comprised”, are not intended to exclude further additives, components, integers or steps. The terms “including” and “comprising” may be used interchangeably. As used herein, the phrases “selected from the group consisting of”, “chosen from”, and the like, include mixtures of the specified materials. Where a numerical limit or range is stated herein, the endpoints are included. Also, all values and subranges within a numerical limit or range are specifically included as if explicitly written herein. References to an element in the singular is not intended to mean “one and only one” unless specifically stated, but rather “one or more”. Unless specifically stated otherwise, terms such as “some” refer to one or more, and singular terms such as “a”, “an” and “the” refer to one or more.

The term “oligopeptide” is used to refer to a peptide with fewer members of amino acids as opposed to a polypeptide or protein. Oligopeptides described herein, are typically comprised of about two to about forty amino acid residues. Oligopeptides include dipeptides (two amino acids), tripeptides (three amino acids), tetrapeptides (four amino acids), pentapeptides (five amino acids), hexapeptides (six amino acids), heptapeptides (seven amino acids), octapeptides (eight amino acids), nonapeptides (nine amino acids), decapeptides (ten amino acids), undecapeptides (eleven amino acids), dodecapeptides (twelve amino acids), icosapeptides (twenty amino acids), tricontapeptides (thirty amino acids), tetracontapeptides (forty amino acids), and the like. Oligopeptides may also be classified according to molecular structure: aeruginosas, cyanopeptolins, microcystins, microviridins, microginins, anabaenopeptins and cyclamides, and the like. Homo-oligopeptides are oligopeptides comprising the same amino acid. In certain embodiments, homo-oligopeptides comprise 10 amino acid poly-valine, poly-alanine, and poly-glycine hexamers.

The meaning of the term “peptides” are defined as small proteins of two or more amino acids linked by the carboxyl group of one to the amino group of another. Accordingly, at its basic level, peptide synthesis of whatever type comprises the repeated steps of adding amino acid or peptide molecules to one another or to an existing peptide chain. The term “peptide” generally has from about 2 to about 100 amino acids, whereas a polypeptide or protein has about 100 or more amino acids, up to a full length sequence which may be translated from a gene. Additionally, as used herein, a peptide can be a subsequence or a portion of a polypeptide or protein. In some embodiments, the peptide consists of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues. In some embodiments, the peptide is from about 30 to about 100 amino acids in length. In certain embodiments, the peptide is from about 40 to about 100 amino acids in length.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the term “prodrug” is intended to encompass proteins, e.g., therapeutic biologics which, under physiologic conditions, are converted into the therapeutically active biologics of the present disclosure. A common method for making a prodrug is to include one or more selected moieties which are hydrolyzed under physiologic conditions to reveal the desired molecule. In some embodiments, the prodrug is converted by an enzymatic activity of the host animal. For example, esters or carbonates (e.g., esters or carbonates of alcohols or carboxylic acids) are preferred prodrugs of the present disclosure. In certain embodiments, some or all of the molecules in a composition represented above can be replaced with the corresponding suitable prodrug, e.g., wherein a hydroxyl in the parent molecule is presented as an ester or a carbonate or carboxylic acid present in the parent protein is presented as an ester.

The meaning of the term “protein” is defined as a linear polymer built from about 20 different amino acids. The type and the sequence of amino acids in a protein are specified by the DNA that produces them. In certain embodiments, the sequences can be natural and unnatural. The sequence of amino acids determines the overall structure and function of a protein. In some embodiments, proteins can contain 50 or more residues. In certain embodiments, proteins can contain greater than about 101 residues in length. A protein's net charge can be determined by two factors: 1) the total count of acidic amino acids vs. basic amino acids; and 2) the specific solvent pH surroundings, which expose positive or negative residues. As used herein, “net positively or net negatively charged proteins” are proteins that, under non-denaturing pH surroundings, have a net positive or net negative electric charge. In general, those skilled in the art will recognize that all proteins may be considered “net negatively charged proteins”, regardless of their amino acid composition, depending on their pH and/or solvent surroundings. For example, different solvents can expose negative or positive side chains depending on the solvent pH. Proteins or peptides are preferably selected from any type of enzyme or antibodies or fragments thereof showing substantially the same activity as the corresponding enzyme or antibody. Proteins or peptides may serve as a structural material (e.g. keratin), as enzymes, as hormones, as transporters (e.g. hemoglobin), as antibodies, or as regulators of gene expression. Proteins or peptides are required for the structure, function, and regulation of cells, tissues, and organs. In some embodiments, the protein is a therapeutic biologic. In certain embodiments, the protein is bovine serum albumin (BSA) or human serum albumin (HSA).

The term “substantially” as used herein, refers to a majority of, or mostly, as in at least about 50%, 60%, 70%, 80%, 90%, 95%, 96%, 97%, 98%, 99%, 99.5%, 99.9%, 99.99%, or at least about 99.999% or more.

It is understood that the specific order or hierarchy of steps in the methods or processes disclosed is an illustration of exemplary approaches. Based upon design preferences, it is understood that the specific order or hierarchy of steps in the methods or processes may be rearranged. Some of the steps may be performed simultaneously. The accompanying methods claims present elements of the various steps in a sample order, and are not meant to be limited to a specific hierarchy or order presented. A phrase such as “embodiment” does not imply that such embodiment applies to all configurations of the subject technology. A disclosure relating to an embodiment may apply to all embodiments, or one or more embodiments. A phrase such as an embodiment may refer to one or more embodiments and vice-versa.

### Particles

Unless otherwise defined, all terms of art, notations and other scientific terminology used herein, are intended to have the meanings commonly understood by those of skill in the art to which this disclosure pertains. In some cases, terms with commonly understood meanings are defined herein, for clarity and/or for ready reference, and the inclusion of such definitions herein, should not necessarily be construed to represent a substantial difference over what is generally understood in the art. The techniques and procedures described or referenced herein, are generally well understood and commonly employed using conventional methodology by those skilled in the art. As appropriate, procedures involving the use of commercially available kits and reagents are generally carried out in accordance with manufacturer defined protocols and/or parameters unless otherwise noted. As used herein, the phrase “and/or” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The terms “particle” or “particles” or “microparticle” or “microparticles” are used herein, interchangeably in the broadest sense, refers to a discrete body or bodies. The particles described herein, are circular, spheroidal and of controlled dispersity with a characteristic size from sub-micrometers to tens of micrometers, in contrast to, e.g., a porous monolithic “cake”, which is typically produced during conventional lyophilization. This morphology allows for a flowable powder (as described by low Hausner ratios) without post-processing. In some embodiments, the term “particle” refers to a quantity of a protein or proteins, e.g., therapeutic biologic or therapeutic biologics which is either in a state of matter that is substantially solid as compared to a liquid droplet or in a gel form. The term “proto-particle” refers to a stage of particle formation in which one or more of the components comprising the particle are in an at least a partial state of desiccation. The total liquid content of the proto-particle is less than that of the droplet and greater than that of the formed particle. Similarly, the average concentration of the solutes is higher than that of the drop but typically less than that of the formed particle. The term “encapsulant” refers to a substance that can be dried or gelled around a particle core to form a shell.

As disclosed herein, a therapeutic biologic, also known as a biologic medical product, or biopharmaceutical, is any pharmaceutical drug product manufactured in, extracted from or semisynthesized from biological sources. Therapeutic biologics can include a wide range of products such as vaccines, blood and blood components, allergenics, somatic cells, gene therapy, tissues, and recombinant therapeutic proteins. In some embodiments, the biologics can be composed of sugars, proteins, or nucleic acids or complex combinations of these substances, or may be living entities such as cells and tissues. Biologics can be isolated from a variety of natural sources, e.g., a human, animal, or microorganism, and may be produced by biotechnology methods or other technologies. Gene-based and cellular biologics, for example, are often used to treat a variety of medical conditions for which no other treatments are available. In some embodiments of the disclosure, the therapeutic biologic is an antibody or fragment thereof. In certain embodiments, the antibody is a human antibody, e.g., human IgG, or a monoclonal antibody (mAb). In some embodiments, the antibody is Rituximab or Trastuzumab. In certain embodiments, the therapeutic biologic is a fragment of an antibody.

The terms “antibody” and “immunoglobulin” are used interchangeably in the broadest sense and include monoclonal antibodies, polyclonal antibodies, multivalent antibodies, and multispecific antibodies, regardless of how they are produced, i.e., using immunization, recombinant, synthetic methodologies. Antibodies can be gamma globulin proteins that are found in blood, or other bodily fluids of vertebrates that function in the immune system to bind antigen, hence identifying and/or neutralizing foreign objects. Antibodies can be assigned to different classes or isotypes. There are five classes of immunoglobulins: IgA, IgD, IgE, IgG, and IgM, having heavy chains designated alpha, delta, epsilon, gamma, and mu, respectively. The gamma class is further divided into subclasses based on the differences in sequences and function, e.g., humans express the following subclasses: IgG1, IgG2, IgG3, IgG4, IgA1, and IgA2. In certain embodiments of the disclosure, the IgG antibody is an IgG1 antibody. In certain embodiments of the disclosure, the IgG antibody is a monoclonal IgG antibody. The L chain from any vertebrate species can be assigned to one of two clearly distinct types, e.g., kappa and lambda, based on the amino acid sequences of their constant domains.

The recognized immunoglobulin genes include the kappa, lambda, alpha, gamma, delta, epsilon and mu constant region genes, as well as the myriad immunoglobulin variable region genes. In some embodiments, light chains are classified as either kappa or lambda. In some embodiments, heavy chains are classified as gamma, mu, alpha, delta, or epsilon, which in turn define the immunoglobulin classes, IgG, IgM, IgA, IgD and IgE, respectively. In certain embodiments of the disclosure, the antibody is an IgG antibody.

An exemplary antibody (immunoglobulin) structural unit comprises a tetramer. Each tetramer is composed of two identical pairs of polypeptide chains, each pair having one “light” (about sss25 kD) and one “heavy” chain (about 50-70 kD). The N-terminus of each chain defines a variable region of about 100 to 110 or more amino acids primarily responsible for antigen recognition. The terms “variable light” chain, domain, region and component are used interchangeably, are abbreviated by “VL” or “VL” and refer to the light chain of an antibody or antibody fragment. Similarly, terms “variable heavy” chain, domain, region and component are used interchangeably, are abbreviated by “VH” or “VH” and refer to the heavy chain of an antibody or antibody fragment. Antibodies are generally a heterotetrameric glycoprotein composed of two identical light (L) chains and two identical heavy (H) chains. Each L chain is linked to a H chain by one covalent disulfide bond. The two H chains are linked to each other by one or more disulfide bonds depending on the H chain isotype. Each H and L chain also has regularly spaced intra-chain disulfide bridges. H and L chains define specific Ig domains. In particular, each H chain has at the N-terminus, a variable domain (VH) followed by three constant domains (CH) for each of the alpha and gamma chains and four CH domains for p and c isotypes. Each L chain has at the N-terminus, a variable domain (VL) followed by a constant domain (CL) at its other end. The VL is aligned with the VH and the CL is aligned with the first constant domain of the heavy chain (CHL). The constant domain includes the Fc portion which comprises the carboxy-terminal portions of both H chains held together by disulfides. The effector functions of antibodies such as ADCC are determined by sequences in the Fc region, which is also the part recognized by Fc receptors (FcR) found on certain types of cells.

As disclosed herein, the pairing of a VH and VL together form a “variable region” or “variable domain” including the amino-terminal domains of the heavy or light chain of the antibody. The variable domain of the heavy chain may be referred to as “VH”. The variable domain of the light chain may be referred to as “VL”. The V domain contains an “antigen binding site” which affects antigen binding and defines specificity of a particular antibody for its particular antigen. V regions span about 110 amino acid residues and consist of relatively invariant stretches called framework regions (FRs) (generally about 4) of 15-30 amino acids separated by shorter regions of extreme variability called “hypervariable regions” (generally about 3) that are each generally 9-12 amino acids long. The FRs largely adopt a p-sheet configuration and the hypervariable regions form loops connecting, and in some cases forming part of, the p-sheet structure. In certain embodiments, the “hypervariable region” refers to the regions of an antibody variable domain which are hypervariable in sequence and/or form structurally defined loops. Generally, antibodies comprise six hypervariable regions; three in the VH (H1, H2, H3), and three in the VL (L1, L2, L3). “Framework” or “FR” residues are those variable domain residues other than the hypervariable region residues defined herein.

The terms “full length antibody”, “intact antibody” and “whole antibody” are used herein, interchangeably, to refer to an antibody in its substantially intact form, not as antibody fragments as defined above. The terms particularly refer to an antibody with heavy chains that contain the Fc region. A full length antibody can be a native sequence antibody or an antibody variant. In certain embodiments, an “intact” or “whole” antibody is one which comprises an antigen-binding site as well as a CL and at least heavy chain constant domains, CH1, CH2 and CH3. The constant domains may be native sequence constant domains, e.g., human native sequence constant domains, or amino acid sequence variants thereof.

As indicated above, the term antibody as used herein, unless otherwise stated or clearly contradicted by context, includes fragments of an antibody that retain the ability to specifically interact, such as bind, to the antigen. It has been shown that the antigen-binding function of an antibody may be performed by fragments of a full-length antibody. Examples of binding fragments encompassed within the term “antibody” include (i) a Fab′ or Fab fragment, a monovalent fragment consisting of the light chain variable domain (VL), heavy chain variable domain (VH), light chain constant region (CL) and heavy chain constant region domain 1 (CH1) domains, or a monovalent antibody as described in WO 2007/059782; (ii) F(ab′)2 fragments, bivalent fragments comprising two Fab fragments linked by a disulfide bridge at the hinge region; (iii) an Fd fragment consisting essentially of the VH and CH1 domains; (iv) an Fv fragment consisting essentially of the VL and VH domains of a single arm of an antibody, (v) a dAb fragment Ward et al., Nature 341, 544-546 (1989), which consists essentially of a VH domain and is also called domain antibody Holt et al; Trends Biotechnol. 2003 November; 21(11):484-90; (vi) camelid or nanobodies Revets et al; Expert Opin Biol Ther. 2005 January; 5(1):111-24 and (vii) an isolated complementarity determining region (CDR). Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Revets et al; Expert Opin Biol Ther. 2005 January; 5(1): 111-24 and Bird et al., Science 242, 423-426 (1988). Such single chain antibodies are encompassed within the term antibody unless otherwise noted or clearly indicated by context. Although such fragments are generally included within the meaning of antibody, they collectively and each independently are unique features of the present invention, exhibiting different biological properties and utility. These and other useful antibody fragments in the context of the present invention are discussed further herein.

As disclosed herein, “whole antibody fragments including a variable domain” include Fab, Fab′, F(ab′)₂, and Fv fragments; diabodies; linear antibodies, single-chain antibody molecules; and multispecific antibodies formed from antibody fragments. The “Fab fragment” consists of an entire L chain along with the variable region domain of the H chain (VH), and the first constant domain of one heavy chain (CHI). Each Fab fragment is monovalent with respect to antigen binding, i.e., it has a single antigen-binding site. A “Fab′ fragment” differs from Fab fragments by having additional few residues at the carboxy terminus of the CHI domain including one or more cysteines from the antibody hinge region. Fab′-SH is the designation herein for Fab′ in which the cysteine residue(s) of the constant domains bear a free thiol group. A “F(ab′)₂ fragment” roughly corresponds to two disulfide linked Fab fragments having divalent antigen-binding activity and is still capable of cross-linking antigen. An “Fv” is the minimum antibody fragment which contains a complete antigen-recognition and binding site. This fragment consists of a dimer of one heavy and one light chain variable region domain in tight, non-covalent association. In a single-chain Fv (scFv) species, one heavy and one light chain variable domain can be covalently linked by a flexible peptide linker such that the light and heavy chains can associate in a “dimeric” structure analogous to that in a two-chain Fv species. From the folding of these two domains emanate six hypervariable loops (3 loops each from the H and L chain) that contribute the amino acid residues for antigen binding and confer antigen binding specificity to the antibody. “Single-chain Fv” also abbreviated as “sFv” or “scFv” are antibody fragments that comprise the VH and VL antibody domains connected to form a single polypeptide chain. In certain embodiments, the scFv polypeptide further comprises a polypeptide linker between the VH and VL domains which enables the scFv to form the desired structure for antigen binding. In some embodiments, a “single variable domain” is half of an Fv (comprising only three CDRs specific for an antigen) that has the ability to recognize and bind antigen, although at a lower affinity than the entire binding site.

In some embodiments, “diabodies” refer to antibody fragments with two antigen-binding sites, which fragments comprise a heavy-chain variable domain (VH) connected to a light-chain variable domain (VL) in the same polypeptide chain (VH-VL). The small antibody fragments are prepared by constructing sFv fragments with short linkers (about 5-10 residues) between the VH and VL domains such that inter-chain but not intra-chain pairing of the V domains is achieved, resulting in a bivalent fragment, i.e., fragment having two antigen-binding sites. In some embodiments, diabodies may be bivalent or bispecific. In certain embodiments, bispecific diabodies are heterodimers of two “crossover” sFv fragments in which the VH and VL domains of the two antibodies are present on different polypeptide chains. Triabodies and tetrabodies are also generally known in the art.

“Antigen binding fragments” of antibodies as described herein, comprise only a portion of an intact antibody, generally including an antigen binding site of the intact antibody and thus retaining the ability to bind antigen. Exemplary examples of antibody fragments encompassed by the present definition include but are not limited to: (i) the Fab fragment, having VL, CL, VH and CHI domains; (ii) the Fab′ fragment, which is a Fab fragment having one or more cysteine residues at the C-terminus of the CHI domain; (iii) the Fd fragment having VH and CHI domains; (iv) the Fd′ fragment having VH and CHI domains and one or more cysteine residues at the C-terminus of the CHI domain; (v) the Fv fragment having the VL and VH domains of a single arm of an antibody; (vi) the dAb fragment which consists of a VH domain; (vii) isolated CDR regions; (viii) F(ab′)₂ fragments, a bivalent fragment including two Fab′ fragments linked by a disulfide bridge at the hinge region; (ix) single chain antibody molecules, e.g. single chain Fv; scFv; (x) “diabodies” with two antigen binding sites, comprising a heavy chain variable domain (VH) connected to a light chain variable domain (VL) in the same polypeptide chain; (xi) “linear antibodies” comprising a pair of tandem Fd, segments (VH-CH1-VH-CH1) which, together with complementary light chain polypeptides, form a pair of antigen binding regions. In some embodiments, an “antigen binding site” generally refers to a molecule that includes at least the hypervariable and framework regions that are required for imparting antigen binding function to a V domain. An antigen binding site may be in the form of an antibody or an antibody fragment, (such as a dAb, Fab, Fd, Fv, F(ab′)₂ or scFv) in a method described herein. In some embodiments, an antigen-binding fragment competes with intact antibody, e.g., with the intact antibody from which the fragment was derived, for antigen binding.

The term “fragment” refers to a part or portion of an antibody or antibody chain comprising fewer amino acid residues than an intact or complete antibody or antibody chain. As used herein, the term “fragment” of an antibody molecule includes Fc fragments and antigen-binding fragments of antibodies, for example, an antibody light chain variable domain (VL), an antibody heavy chain variable domain (VH), a single chain antibody (scFv), a F(ab′)2 fragment, a Fab fragment, an Fd fragment, an Fv fragment, a single domain antibody fragment (DAb), a one-armed (monovalent) antibody, or any antigen-binding molecule formed by combination, assembly or conjugation of such antigen binding fragments.

In some embodiments, the term “single-chain Fv” or “scFv” or “single chain” antibody can refer to antibody fragments comprising the VH and VL domains of antibody, wherein these domains are present in a single polypeptide chain. Generally, the Fv polypeptide further comprises a polypeptide linker between the VH and VL domains which enables the sFv to form the desired structure for antigen binding. For a review of sFv, see Pluckthun, THE PHARMACOLOGY OF MONOCLONAL ANTIBODIES, vol. 113, Rosenburg and Moore eds. Springer-Verlag, New York, pp. 269-315 (1994).

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies (mAbs) are highly specific, being directed against a single antigenic site or determinant on the antigen. In addition to their specificity, the monoclonal antibodies are advantageous in that they may be synthesized uncontaminated by other antibodies. Monoclonal antibodies may be prepared by the hybridoma methodology. The monoclonal antibodies may also be isolated from phage antibody libraries using molecular engineering techniques. The monoclonal antibodies of the disclosure may be generated by recombinant DNA methods, and are sometimes referred to as “recombinant antibodies” or “recombinant monoclonal antibodies” as described herein. In some embodiments, a monoclonal antibody is a single species of antibody wherein every antibody molecule recognizes the same epitope because all antibody producing cells are derived from a single B-lymphocyte cell line. The methods for generating monoclonal antibodies (mAbs) generally begin along the same lines as those for preparing polyclonal antibodies. In some embodiments, rodents such as mice and rats are used in generating monoclonal antibodies. In some embodiments, rabbit, sheep, or frog cells are used in generating monoclonal antibodies. The use of rats is well known and may provide certain advantages. Mice, e.g., BALB/c mice, are routinely used and generally give a high percentage of stable fusions. In certain embodiments of the disclosure, the antibody is a monoclonal antibody. In certain embodiments of the disclosure, the IgG antibody is monoclonal.

In some embodiments, recombinant antibody fragments may be isolated from phage antibody libraries using techniques well known in the art. See, for example, Clackson et al., 1991, Nature 352: 624-628; Marks et al., 1991, J. Mol. Biol. 222: 581-597. Recombinant antibody fragments may be derived from large phage antibody libraries generated by recombination in bacteria (Sblattero and Bradbury, 2000, Nature Biotechnology 18:75-80; and as described herein). Polynucleotides encoding the VH and VL components of antibody fragments, i.e., scFv, may be used to generate recombinant full length immunoglobulins using methods known in the art (see, for example, Persic et al., 1997, Gene 187: 9-18).

An “isolated antibody” is one that has been identified and separated and/or recovered from a component of its pre-existing environment. Contaminant components are materials that would interfere with therapeutic uses for the antibody, and may include enzymes, hormones, and other proteinaceous or nonproteinaceous solutes.

As used herein, a “human antibody” refers to an antibody that possesses an amino acid sequence that corresponds to that of an antibody produced by a human. Human antibodies can be produced using various techniques known in the art, including phage-display libraries. Human antibodies can be prepared by administering the antigen to a transgenic animal that has been modified to produce such antibodies in response to antigenic challenge, but whose endogenous loci has been disabled. “Humanized” forms of non-human, e.g., rodent, antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some embodiments, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

An “affinity matured” antibody is one with one or more alterations in one or more hypervariable region thereof that result in an improvement in the affinity of the antibody for antigen, compared to a parent antibody that does not possess those alterations. In some embodiments, affinity matured antibodies can have micromolar affinities for the target antigen. In some embodiments, affinity matured antibodies can have nanomolar or even picomolar affinities for the target antigen. Affinity matured antibodies are produced by procedures known in the art.

In some embodiments of the foregoing methods, the protein, e.g., therapeutic biologic is an antibody. In some embodiments, the antibody includes but are not limited to 3F8, Abagovomab, Abatacept, Abciximab, Abituzumab, Abrezekimab, Abrilumab, Acritumomab, Actoxumab, Abituzumab, Adalimumab-adbm, Adalimumab-atto, Adalimumab-bwwb, Adecatumumab, Ado-trastuzumab emtansine, Aducanumab, Afasevikumab, Afelimomab, Aflibercept, Afutuzumab, Alacizumab pegol, ALD518, Alefacept, Alemtuzumab, Alirocumab, Altumomab pentetate, Amatuximab, Anatumomab mafenatox, Andecaliximab, Anetumab ravtansine, Anifrolumab, Anrukinzumab, Ansuvimab, Apolizumab, Aprutumab ixadotin, Arcitumomab, Ascrinvacumab, Aselizumab, Atezolizumab, Atidortoxumab, Atinumab, Atlizumab, Atoltivimab, Atorolimumab, Avelumab, Azintuxizumab vedotin, Bapineuzumab, Basiliximab, Bavituximab, BCD-100, Bectumomab, Begelomab, Belantamab mafodotin, Belatacept, Belimumab, Bemarituzumab, Benralizumab, Bermekimab, Bersanlimab, Bertilimumab, Besilesomab, Bevacizumab, Bevacizumab-awwb, Bezlotoxumab, Biciromab, Bimagrumab, Bimekizumab, Birtamimab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Camidanlumab tesirine, Camrelizumab, Canakinumab, Cantuzumab mertansine, Cantuzumab ravtansine, Caplacizumab, Capromab pendetide, Carlumab, Carotuximab, Catumaxomab, cBR96-doxorubicin immunoconjugate, Cedelizumab, Cemiplimab, Cergutuzumab amunaleukin, Cergutuzumab amunaleukin, Certolizumab pegol, Cetrelimab, Cetuximab, Cibisatamab, Cirmtuzumab, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, CR6261, Crizanlizumab, Crotedumab, Cusatuzumab, Dacetuzumab, Daclizumab, Dalotuzumab, Dapirolizumab pegol, Daratumumab, Dectrekumab, Demcizumab, Denileukin diftitox, Denintuzumab mafodotin, Denosumab, Depatuxizumab mafodotin, Derlotuximab biotin, Detumomab, Dezamizumab, Dinutuximab, Diridavumab, Domagrozumab, Dorlimomab aritox, Dostarlimab, Drozitumab, DS-8201, Duligotumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emibetuzumab, Emicizumab, Enapotamab vedotin, Enavatuzumab, Enfortumab vedotin, Enlimomab pegol, Enoblituzumab, Enokizumab, Enoticumab, Ensituximab, Epitumomab cituxetan, Epoetin-alfa, Epoetin-alfa-epbx, Epratuzumab, Eptinezumab, Erenumab, Erlizumab, Ertumaxomab, Etanercept, Etanercept-szzs, Etaracizumab, Etigilimab, Etrolizumab, Evinacumab, Evolocumab, Exbivirumab, Factor VIII Fc fusion protein, Factor IX Fc fusion protein, Fanolesomab, Faralimomab, Faricimab, Farletuzumab, Fasinumab, Felvizumab, Fezakinumab, Fibatuzumab, Ficlatuzumab, Figitumumab, Filgrastim, Filgrastim-sndz, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, Ibalizumab, IBI308, Ibritumomab tiuxetan, Icrucumab, Idarucizumab, Ifabotuzumab, Igovomab, Iladatuzumab vedotin, IMAB362, Imalumab, Imaprelimab, Imciromab, Imgatuzumab, Inclacumab, Indatuximab ravtansine, Indusatumab vedotin, Inebilizumab, Infliximab, Infliximab-abda, Infliximab-dyyb, Infliximab-qbtx, Intetumumab, Inolimomab, Inotuzumab ozogamicin, Ipilimumab, Iomab-B, Iratumumab, Isatuximab, Iscalimab, Istiratumab, Itolizumab, Ixekizumab, Keliximab, Labetuzumab, Lacnotuzumab, Ladiratuzumab vedotin, Lambrolizumab, Lampalizumab, Lanadelumab, Landogrozumab, Laprituximab emtansine, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Loncastuximab tesirine, Losatuxizumab vedotin, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lupartumab amadotin, Lutikizumab, Maftivimab, Mapatumumab, Margetuximab, Marstacimab, Maslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Naratuximab emtansine, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odesivimab, Odesivimab-ebgn, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Ozoralizumab, Pagibaximab, Palivizumab, Pamrevlumab, Panitumumab, Pankomab, Panobacumab, Parsatuzumab, Pascolizumab, Pasotuxizumab, Pateclizumab, Patritumab, PDR001, Pegfilgrastim-jmdb, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Porgaviximab, Prasinezumab, Prezalizumab, Priliximab, Pritoxaximab, Pritumumab, PRO 140, Quilizumab, Racotumomab, Radretumab, Rafivirumab, Ralpancizumab, Ramucirumab, Ranevetmab, Ranibizumab, Raxibacumab, Ravagalimab, Ravulizumab, Refanezumab, Regavirumab, Relatlimab, Remtolumab, Reslizumab, Rilonacept, Rilotumumab, Rinucumab, Risankizumab, Rituximab, Rituximab-abbs, Rituximab-arrx, Rituximab-pvvr, Rivabazumab pegol, Rivabazumab pegol, Robatumumab, Rmab, Roledumab, Romilkimab, Romiplostim, Romosozumab, Rontalizumab, Rosmantuzumab, Rovalpituzumab tesirine, Rovalpituzumab tesirine, Rovelizumab, Rozanolixizumab, Ruplizumab, Sacituzumab govitecan, Samalizumab, Samrotamab vedotin, Sapelizumab, Sarilumab, Satralizumab (SA237), Satumomab pendetide, Secukinumab, Selicrelumab, Seribantumab, Setoxaximab, Setrusumab, Sevirumab, Sibrotuzumab, SGN-CD19A, SGN-CD33A, SHP647, Sifalimumab, Siltuximab, Simtuzumab, Siplizumab, Sirtratumab vedotin, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Tafasitamab, Talacotuzumab, Talizumab, Tamtuvetmab, Tanezumab, Taplitumomab paptox, Tarextumab, Tavolimab, Tefibazumab, Telimomab aritox, Telisotuzumab vedotin, Tenatumomab, Teneliximab, Teplizumab, Tepoditamab, Teprotumumab, Tesidolumab, Tetulomab, Tezepelumab, TGN1412, Tibulizumab, Ticilimumab, Tildrakizumab, Tigatuzumab, Timigutuzumab, Timolumab, Tiragotumab, Tislelizumab, Tisotumab vedotin, TNX-650, Tocilizumab, Tomuzotuximab, Toralizumab, Tosatoxumab, Tositumomab, Tovetumab, Tralokinumab, Trastuzumab, Trastuzumab-anns, Trastuzumab-dkst, Trastuzumab-dttb, Trastuzumab emtansine, Trastuzumab-pkrb, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vadastuximab talirine, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Xentuzumab, XMAB-5574, Zalutumumab, Zanolimumab, Zatuximab, Zenocutuzumab, Ziralimumab, Zolbetuximab (IMAB362, Claudiximab), Ziv-aflibercept, or Zolimomab aritox.

In some embodiments of the foregoing methods, the antibody is monoclonal. In certain embodiments, the monoclonal antibody includes but are not limited to 3F8, 8H9, Abatacept, Abagovomab, Abciximab, Abituzumab, Adalimumab-adbm, Adalimumab-atto, Adalimumab-bwwb, Abrilumab, Actoxumab, Abituzumab, Abrezekimab, Abrilumab, Actoxumab, Adalimumab, Adecatumumab, Ado-trastuzumab emtansine, Aducanumab, Afasevikumab, Afelimomab, Aflibercept, Afutuzumab, Alacizumab pegol, ALD518, Alefacept, Alemtuzumab, Alirocumab, Altumomab pentetate, Amatuximab, Anatumomab mafenatox, Andecaliximab, Anetumab ravtansine, Anifrolumab, Anrukinzumab (IMA-638), Ansuvimab, Apolizumab, Arcitumomab, Ascrinvacumab, Aselizumab, Atezolizumab, Atidortoxumab, Atinumab, Atlizumab (tocilizumab), Atoltivimab, Atorolimumab, Avelumab, Bapineuzumab, Basiliximab, Bevacizumab, Bevacizumab-awwb, BCD-100, Bectumomab, Begelomab, Belatacept, Belimumab, Bemarituzumab, Benralizumab, Bermekimab, Bersanlimab, Bertilimumab, Besilesomab, Bezlotoxumab, Biciromab, Bimagrumab, Bimekizumab, Birtamimab, Bivatuzumab mertansine, Bleselumab, Blinatumomab, Blontuvetmab, Blosozumab, Bococizumab, Brazikumab, Brentuximab vedotin, Briakinumab, Brodalumab, Brolucizumab, Brontictuzumab, Burosumab, Cabiralizumab, Camrelizumab, Canakinumab, Cantuzumab mertansine, Cantuzumab ravtansine, Caplacizumab, Capromab pendetide, Carlumab, Carotuximab, Catumaxomab, Cedelizumab, Cemiplimab, Certolizumab pegol, Cetrelimab, Cetuximab, Cibisatamab, Cirmtuzumab, Ch.14.18, Citatuzumab bogatox, Cixutumumab, Clazakizumab, Clenoliximab, Clivatuzumab tetraxetan, Codrituzumab, Cofetuzumab pelidotin, Coltuximab ravtansine, Conatumumab, Concizumab, Cosfroviximab, Crenezumab, CR6261, Crizanlizumab, Crotedumab, Cusatuzumab, Dacetuzumab, Daclizumab, Dalotuzumab, Dapirolizumab pegol, Daratumumab, Dectrekumab, Demcizumab, Denileukin diftitox, Denintuzumab mafodotin, Denosumab, Derlotuximab biotin, Detumomab, Dezamizumab, Dinutuximab, Diridavumab, Domagrozumab, Dorlimomab aritox, Dostarlimab, Drozitumab, Duligotumab, Dupilumab, Durvalumab, Dusigitumab, Duvortuxizumab, Ecromeximab, Eculizumab, Edobacomab, Edrecolomab, Efalizumab, Efungumab, Eldelumab, Elezanumab, Elgemtumab, Elotuzumab, Elsilimomab, Emactuzumab, Emibetuzumab, Emicizumab, Enavatuzumab, Enfortumab vedotin, Enlimomab pegol, Enoblituzumab, Enokizumab, Enoticumab, Ensituximab, Epitumomab cituxetan, Epoetin-alfa, Epoetin-alfa-epbx, Epratuzumab, Eptinezumab, Erenumab, Erlizumab, Ertumaxomab, Etanercept, Etanercept-szzs, Etaracizumab, Etigilimab, Etrolizumab, Evinacumab, Evolocumab, Exbivirumab, Factor VIII Fc fusion protein, Factor IX Fc fusion protein, Fanolesomab, Faralimomab, Faricimab, Farletuzumab, Fasinumab, FBTA05, Felvizumab, Fezakinumab, Fibatuzumab, Ficlatuzumab, Figitumumab, Filgrastim, Filgrastim-sndz, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab, Galcanezumab, Galiximab, Ganitumab, Gantenerumab, Gatipotuzumab, Gavilimomab, Gedivumab, Gemtuzumab ozogamicin, Gevokizumab, Gilvetmab, Gimsilumab, Girentuximab, Glembatumumab vedotin, Golimumab, Gomiliximab, Gosuranemab, Guselkumab, Ibalizumab, IBI308, Ibritumomab tiuxetan, Icrucumab, Idarucizumab, Ifabotuzumab, Igovomab, IMAB362, Imalumab, Imaprelimab, Imciromab, Imgatuzumab, Inclacumab, Indatuximab ravtansine, Indusatumab vedotin, Inebilizumab, Infliximab, Infliximab-abda, Infliximab-dyyb, Infliximab-qbtx, Intetumumab, Inolimomab, Inotuzumab ozogamicin, Ipilimumab, Iratumumab, Isatuximab, Iscalimab, Istiratumab, Itolizumab, Ixekizumab, Keliximab, Labetuzumab, Lacnotuzumab, Lambrolizumab, Lampalizumab, Lanadelumab, Landogrozumab, Larcaviximab, Lebrikizumab, Lemalesomab, Lendalizumab, Lenvervimab, Lenzilumab, Lerdelimumab, Leronlimab, Lesofavumab, Letolizumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Lutikizumab, Maftivimab, Mapatumumab, Margetuximab, Marstacimab, Maslimomab, Mavrilimumab, Matuzumab, Mepolizumab, Metelimumab, Milatuzumab, Minretumomab, Mirikizumab, Mirvetuximab soravtansine, Mitumomab, Modotuximab, Mogamulizumab, Monalizumab, Morolimumab, Mosunetuzumab, Motavizumab, Moxetumomab pasudotox, Muromonab-CD3, Nacolomab tafenatox, Namilumab, Naptumomab estafenatox, Narnatumab, Natalizumab, Navicixizumab, Navivumab, Naxitamab, Nebacumab, Necitumumab, Nemolizumab, NEOD001, Nerelimomab, Nesvacumab, Netakimab, Nimotuzumab, Nirsevimab, Nivolumab, Nofetumomab merpentan, Obiltoxaximab, Obinutuzumab, Ocaratuzumab, Ocrelizumab, Odesivimab, Odesivimab-ebgn, Odulimomab, Ofatumumab, Olaratumab, Oleclumab, Olendalizumab, Olokizumab, Omalizumab, Omburtamab, OMS721, Onartuzumab, Ontuxizumab, Onvatilimab, Opicinumab, Oportuzumab monatox, Oregovomab, Orticumab, Otelixizumab, Otilimab, Otlertuzumab, Oxelumab, Ozanezumab, Pagibaximab, Palivizumab, Pamrevlumab, Panitumumab, Pankomab, Panobacumab, Parsatuzumab, Pascolizumab, Pasotuxizumab, Pateclizumab, Patritumab, PDR001, Pegfilgrastim-jmdb, Pembrolizumab, Pemtumomab, Perakizumab, Pertuzumab, Pexelizumab, Pidilizumab, Pinatuzumab vedotin, Pintumomab, Placulumab, Plozalizumab, Pogalizumab, Polatuzumab vedotin, Ponezumab, Porgaviximab, Prasinezumab, Prezalizumab, Priliximab, Pritoxaximab, Pritumumab, PRO 140, Quilizumab, Tetulomab, Racotumomab, Radretumab, Rafivirumab, Ralpancizumab, Ramucirumab, Ranevetmab, Ranibizumab, Raxibacumab, Ravagalimab, Ravulizumab, Refanezumab, Regavirumab, Relatlimab, Remtolumab, Reslizumab, Rilonacept, Rilotumumab, Rinucumab, Risankizumab-rzaa, Rituximab, Rituximab-abbs, Rituximab-arrx, Rituximab-pvvr, Robatumumab, Rmab, Roledumab, Romilkimab, Romiplostim, Romosozumab, Rontalizumab, Rosmantuzumab, Rovelizumab, Rozanolixizumab, Ruplizumab, Sacituzumab govitecan, Samalizumab, Sarilumab, Satralizumab (SA237), Satumomab pendetide, Secukinumab, Selicrelumab, Seribantumab, Setoxaximab, Setrusumab, Sevirumab, Sibrotuzumab, SGN-CD19A, SGN-CD33A, SHP647, Sifalimumab, Siltuximab, Simtuzumab, Siplizumab, Sirukumab, Sofituzumab vedotin, Solanezumab, Solitomab, Sonepcizumab, Sontuzumab, Spartalizumab, Stamulumab, Sulesomab, Suptavumab, Sutimlimab, Suvizumab, Suvratoxumab, Tabalumab, Tacatuzumab tetraxetan, Tadocizumab, Tafasitamab, Talacotuzumab, Talizumab, Tamtuvetmab, Tanezumab, Taplitumomab paptox, Tarextumab, Tavolimab, Tefibazumab, Telimomab aritox, Tenatumomab, Teneliximab, Teplizumab, Tepoditamab, Teprotumumab, Tesidolumab, Tetulomab (lilotomab), Tezepelumab, TGN1412, Tibulizumab, Ticilimumab (tremelimumab), Tildrakizumab, Tigatuzumab, Timigutuzumab, Timolumab, Tiragotumab, Tislelizumab, TNX-650, Tocilizumab (atlizumab), Tomuzotuximab, Toralizumab, Tosatoxumab, Tositumomab, Tovetumab, Tralokinumab, Trastuzumab, Trastuzumab-anns, Trastuzumab-dkst, Trastuzumab-dttb, Trastuzumab emtansine, Trastuzumab-pkrb, TRBS07, Tregalizumab, Tremelimumab, Trevogrumab, Tucotuzumab celmoleukin, Tuvirumab, Ublituximab, Ulocuplumab, Urelumab, Urtoxazumab, Ustekinumab, Utomilumab, Vanalimab, Vandortuzumab vedotin, Vantictumab, Vanucizumab, Vapaliximab, Varisacumab, Varlilumab, Vatelizumab, Vedolizumab, Veltuzumab, Vepalimomab, Vesencumab, Visilizumab, Vobarilizumab, Volociximab, Vonlerolizumab, Vopratelimab, Vorsetuzumab mafodotin, Votumumab, Xentuzumab, XMAB-5574, Zalutumumab, Zanolimumab, Zatuximab, Zenocutuzumab, Ziralimumab, Zolbetuximab (IMAB362, Claudiximab), Ziv-aflibercept, Zolimomab aritox or the corresponding anti-drug antibody in a sample from a human patient. In certain embodiments, the monoclonal antibody is Rituximab, Rituximab-abbs, Rituximab-arrx, or Rituximab-pvvr. In some embodiments, the monoclonal antibody is Atoltivimab, Maftivimab, Odesivimab-ebgn, or a combination thereof.

In some embodiments, the monoclonal antibody is a biosimilar. In certain embodiments, the biosimilar includes but are not limited to Adalimumab-adbm, Adalimumab-atto, Adalimumab-bwwb, Bevacizumab-awwb, Epoetin alfa-epbx, Etanercept-szzs, Infliximab-abda, Infliximab-dyyb, Infliximab-qbtx, Filgrastim-sndz, Odesivimab-ebgn, Pegfilgrastim-jmdb, Pegfilgrastim-bmez, Risankizumab-rzaa, Rituximab-abbs, Rituximab-arrx, Rituximab-pvvr, Trastuzumab-anns, Trastuzumab-dttb, Trastuzumab-pkrb, or Trastuzumab-dkst. In some embodiments, the active biosimilar substance is Adalimumab, Bevacizumab, Enoxaparin sodium, Epoetin alfa, Epoetin zeta, Etanercept, Filgrastim, Follitropin alfa, Infliximab, Insulin glargine, Insulin lispro, Pegfilgrastim, Risankizumab, Rituximab, Rituximab-abbs, Rituximab-arrx, Rituximab-pvvr, Somatropin, Teriparatide, or Trastuzumab. In some embodiments, the biosimilar is Rituximab, Rituximab-abbs, Rituximab-arrx, or Rituximab-pvvr. In certain embodiments, the biosimilar is Trastuzumab-anns, Trastuzumab-dttb, Trastuzumab-pkrb, or Trastuzumab-dkst.

In some embodiments, the targeting moiety is an antibody from an intact polyclonal antibody, an intact monoclonal antibody, an antibody fragment, a single chain Fv (scFv) mutant, a multispecific antibody, a bispecific antibody, a chimeric antibody, a humanized antibody, a human antibody, a fusion protein comprising an antigenic determinant portion of an antibody, or other modified immunoglobulin molecules comprising antigen recognition sites.

In certain embodiments, the therapeutic biologic is ledipasvir/sofosbuvir, insulin glargine, lenalidomide, pneumococcal 13-valent conjugate vaccine, fluticasone/salmeterol, elvitegravir/cobicistat/emtricitabine/tenofovir alafenamide, emtricitabine, rilpivirine and tenofovir alafenamide, emtricitabine/tenofovir alafenamide, grazoprevir/elbasvir, coagulation factor VIIa recombinant, epoetin alfa, Aflibercept or etanercept.

In some embodiments, the therapeutic biologic is Abatacept, AbobotulinumtoxinA, Agalsidase beta, Albiglutide, Aldesleukin, Alglucosidase alfa, Alteplase (cathflo activase), Anakinra, Asfotase alfa, Asparaginase, Asparaginase Erwinia chrysanthemi, Becaplermin, Belatacept, Collagenase, Collagenase clostridium histolyticum, Darbepoetin alfa, Denileukin diftitox, Dornase alfa, Dulaglutide, Ecallantide, Elosulfase alfa, Etanercept-szzs, Filgrastim, Filgrastim-sndz, Galsulfase, Glucarpidase, Idursulfase, IncobotulinumtoxinA, Interferon alfa-2b, Interferon alfa-n3, Interferon beta-1a, Interferon beta-1b, Interferon gamma-1b, Laronidase, Methoxy polyethylene glycol-epoetin beta, Metreleptin, Ocriplasmin, OnabotulinumtoxinA, Oprelvekin, Palifermin, Parathyroid hormone, Pegaspargase, Pegfilgrastim, Peginterferon alfa-2a, Peginterferon alfa-2a co-packaged with ribavirin, Peginterferon alfa-2b, Peginterferon beta-1a, Pegloticase, Rasburicase, Reteplase, Rilonacept, RimabotulinumtoxinB, Romiplostim, Sargramostim, Sebelipase alfa, Tbo-filgrastim, Tenecteplase, or Ziv-aflibercept.

The protein, e.g., therapeutic biologic in the particles as disclosed herein, has an activity per unit of about 0.5 to about 1.0, about 0.75 to about 1.0 activity per unit, or about 0.9 to about 1.0 activity per unit. Activity is measured relative to the same protein prior to particle formation. In some embodiments, the protein has an activity per unit of about 0.5 to about 1.0. The term “activity” refers to the ratio of a functional or structural aspect of a protein, e.g., an antibody, antibody fragment, bovine serum albumin (BSA), or human serum albumin (HSA), at two points in time. The denominator of the ratio corresponds to a measure of the functional or structural aspect of the protein in the aqueous feed solution, immediately in advance of droplet formation. The numerator of the ratio corresponds to the same measure of a functional or structural aspect of the protein at a later point in time, e.g., immediately after particle formation.

The particles according to the disclosure are circular. Circularity can serve as an indicator of the shape of the particle. The particles described herein, can have a characteristic circularity, e.g., have a relative shape, that is substantially circular. This characteristic describes and defines the form of a particle on the basis of its circularity. The circularity is 1.0 when the particle has a completely circular structure. Particles according to the disclosure, have a circularity of about 0.80 to about 1.00, 0.90 to about 1.00, 0.95 to about 1.00, 0.96 to about 1.00, 0.97 to about 1.00, 0.98 to about 1.00, or 0.99 to about 1.00. In some embodiments, the circularity of the particles is about 0.85 to about 1.00. In some embodiments, the circularity of the particles about 0.90 to about 1.00. In certain embodiments, the circularity of the particles is about 0.95 to about 1.00. In some embodiments, the circularity of the particles is about 0.98 to about 1.00. In certain embodiments, the circularity of the particles is about 1.00. The diameter and the circularity of the particles can be determined by the processing of an image observed under an electron microscope or the like or a flow-type particle image analyzer. The circularity can also be determined by subjecting particles to circularity measurement and averaging the resulting values. For example, circularity (circ) can be calculated using the following formula:

$$\\ \begin{matrix}circ = 4 * π * \frac{Area}{{Perimeter}^{{}_{} 2}} . & Eq . 1\end{matrix}$$

The term “perimeter”, as used herein, refers to the boundary of a closed plane figure or the sum of all borders of a two-dimensional image. As used herein, the term “area”, refers to the crossectional area of a two-dimensional image of a particle. The circularity of a particle can also be described as the ratio of the smallest dimension of the particle to its largest diameter. For a perfect circle, the ratio is 1. The percentage circularity can be calculated by multiplying the circularity by 100. The circularity can be calculated, for example, by measuring the aspect ratio using any software adapted to deal with images, for example, images obtained by microscopy, in particular, scanning electron microscopy (SEM) or transmission electron microscopy (TEM). In some embodiments, methods of measuring particle circularity include image analysis of scanning electron micrographs of the particles in which the average roundness is calculated on the basis of the cross-sectional shapes of the particles projected onto the plane of the image. Such roundness factors can be extended to identify the corresponding circularity.

In some embodiments of the disclosure, the mixing, e.g., stirring, shaking, vortexing, or sonication, operation may be controlled to provide particles having particular characteristics, such as particles having a substantially smooth surface. “Surface roughness”, as used herein, means a particle having numerous wrinkles or creases, e.g., being ridged or wrinkled. The term “pit”, as used herein, refers to an indentation or crevice in the particle, either an indentation or crevice in the two-dimensional image or an indentation or crevice in an object. The term “spike”, as used herein, refers to a projection pointing outward from the centroid of a particle, a projection pointing outward from the centroid of a two-dimensional image or a sharp projection pointing outward from an object.

In certain embodiments of the disclosure, the particles as described herein, have a surface morphology that is smooth rather than ridged or wrinkled. The surface roughness of the particles may be decreased by controlling the mixing process to form the particles as described herein. In certain embodiments, the mixing conditions can be selected to control the particle morphology in order to enhance the smoothness of the particle's surface. In particular, the mixing conditions can be selected to provide particles having a substantially smooth surface. In certain embodiments, the particles have a substantially smooth surface after mixing the first aqueous liquid comprising the protein, pH adjusting agent with the organic liquid. In some embodiments, the particles have a substantially smooth surface after mixing the first aqueous liquid comprising the protein, pH adjusting agent and one or more excipients with the organic liquid. A person of ordinary skill in the field of this disclosure can readily assess the surface morphology of the disclosed particles using routine and standard techniques.

In some embodiments, the particles have less than about 10% internal void spaces, e.g., less than about 9, 8, 7, 6, 5, 4, 3, 2, or 1% internal void spaces after mixing. In certain embodiments, the particle is substantially free from any internal void spaces after mixing. Suitable methods for determining internal void space can be accomplished by using Focused Ion Beam Scanning Electron Microscopy (FIB-SEM), which can be used to visualize “accessible” and “inaccessible” void spaces, or gas displacement pycnometry (Micromeritics Instrument Corporation of Norcross, Ga), which can determine “accessible” voids (void spaces accessible from the surface rather than those resembling a core-shell structure that are “unaccessible form the surface”). Gas pycnometry is a common analytical technique that uses a gas displacement method to measure volume. Inert gases, such as helium or nitrogen, are used as the displacement medium. True volume is total volume minus volume accessible to the gas. Density is calculated by dividing sample weight with true volume. The sample is sealed in the instrument compartment of a known volume, the appropriate inert gas is admitted, and then expanded into another precision internal volume. The pressure before and after expansion is measured and used to compute the sample volume. Dividing this volume into the sample weight gives the gas displacement density. Cross-sections of typical particles of the disclosure indicate an absence of pores (substantially free from any internal void spaces) and low particle porosity as shown by FIB-SEM or by gas pycnometry using helium at temperatures at about 22° C. to provide densities typically averaging about 1.3 g/cm³ with standard deviations at about 0.0005 g/cm³. For example, internal void space can be calculated using the following formula: internal void space=Aᵥ/Aₚ, where A, is the total area of void spaces and Aₚ is the total area of the particle.

In some embodiments, the particles have less than about 10% internal void spaces. In some embodiments, the particles have less than about 5% internal void spaces. In certain embodiments, the particles have less than about 3% internal void spaces. In some embodiments, the particles have less than about 1% internal void spaces. In certain embodiments, the particles are substantially free from any internal void spaces.

In some embodiments, the particles have a diameter of about 0.1 to about 1000 μm after mixing, e.g., about 0.1 to about 900, 800, 700, 600, 500, 400, 300, 200, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, or about 0.2 μm after mixing. In certain embodiments, the particles have a diameter of about 1 to about 100 μm after mixing, e.g., about 2, 3, 4, 5, 6, 7, 8, 9, 10, 11, 12, 13, 14, 15, 16, 17, 18, 19, 20, 25, 30, 35, 40, 45, or 50 to about 100 μm after mixing. In some embodiments, the particles have a diameter of about 4 to about 100 μm after mixing. In some embodiments, the particles have a diameter of about 10 to about 100 μm after mixing. In certain embodiments, the particles have a diameter of about 20 to about 50 μm after mixing. In some embodiments, the particle is intentionally controlled in its diameter during mixing. In some embodiments, the particles have a diameter of about 0.1 to about 1000 μm, e.g., about 0.1 to about 900, 800, 700, 600, 500, 400, 300, 200, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, or about 0.2 μm. Methods of measuring the particle size and distribution include imaging flow cytometry, laser diffraction, and image analysis of scanning electron micrographs of the particles in which an average spherical radius or diameter can be calculated on the basis of the cross-sectional areas of the particles projected onto the plane of the image.

In some embodiments, the particles have greater than about 60% protein by weight after mixing, e.g., greater than about 65, 70, 75, 80, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% protein by weight after mixing. In some embodiments, the particles have greater than about 70% protein by weight. In certain embodiments, the particles have greater than about 80% protein by weight. In certain embodiments, the particles have greater than about 90% protein by weight. In certain embodiments, the particles have greater than about 95% protein by weight. In some embodiments, the particles have greater than about 98% protein by weight.

The terms “pH adjusting agent” or “buffering species” or “buffering agent” or “buffer” are used herein, interchangeably in the broadest sense, to refer to substances that maintain a constant pH over a given range by neutralizing the effects of hydrogen ions. A pH adjusting agent is generally a solution containing an acid and a base, or a salt, e.g., histidine/histidine·HCl, that tends to maintain a constant hydrogen ion concentration. In some embodiments, the particles comprise a pH adjusting agent. In some embodiments, the particles comprise one or more pH adjusting agents. In certain embodiments, the pH adjusting agent is acetate, citrate, glutamate, glycinate, histidine, lactate, maleate, phosphate, succinate, tartrate, bicarbonate, aluminum hydroxide, phosphoric acid, hydrochloric acid, sulfuric acid, DL-lactic/glycolic acids, phosphorylethanolamine, tromethamine, imidazole, glyclyglycine, monosodium glutamate, sodium hydroxide, potassium hydroxide, sodium phosphate, or a combination thereof. In some embodiments, the pH adjusting agent is acetate, citrate, histidine, phosphate, succinate, hydrochloric acid, sodium hydroxide, or a combination thereof. In certain embodiments, the pH adjusting agent is histidine, hydrochloric acid, or a combination thereof. In some embodiments, the pH adjusting agent is histidine·HCl, sodium succinate, sodium phosphate, or a combination thereof. In some embodiments, the pH levels of the methods as disclosed herein, is about 4.5 to about 7.5. In some embodiments, the pH levels of the methods as disclosed herein, is about 5.0 to about 7.0. In some embodiments, the pH level is about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5. In certain embodiments, the pH level is about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0. In certain embodiments, the pH level is about 5.0, 5.5, 6.0, 6.5, or 7.0.

The term “excipient” refers to an additive to a preparation or formulation, which may be useful in achieving a desired modification to the characteristics of the preparation or particle formulation. Such modifications include, but are not limited to, physical stability, chemical stability, and therapeutic efficacy. Exemplary excipients include, but are not limited to a carbohydrate, a salt, a chelator, a mineral, a polymer, a surfactant, an amino acid, an oligopeptide, a biologic excipient, a chemical excipient, an antiseptic, an antioxidant, a paraben, a bactericide, a fungicide, a vitamin, a preservative, an analgesic, and/or nutrient media. In some embodiments, the particles further comprise one or more excipients. In some embodiments, the one or more different excipients is selected from one or more of a carbohydrate, a salt, a chelator, a mineral, a polymer, a surfactant, a protein stabilizer, an emulsifier, an antiseptic, an amino acid, an antioxidant, an organic solvent, a paraben, a bactericide, a preservative, and an analgesic. In certain embodiments, step ii. further comprises adding one or more different excipients to each container. In certain embodiments, the excipient further comprises at least one pharmaceutically acceptable additive, diluent, carrier, or a combination thereof.

In some embodiments, the carbohydrate may be from the families of monosaccharides, disaccharides, oligosaccharides, or polysaccharides. In some embodiments, the carbohydrate is dextran, trehalose, sucrose, agarose, mannitol, lactose, sorbitol, maltose, starch, alginates, xanthan, galactomanin, agar, agarose, or a combination thereof. In certain embodiments, the carbohydrate is dextran, trehalose, sucrose, agarose, mannitol, lactose, sorbitol, maltose, or a combination thereof. In some embodiments, the carbohydrate is trehalose, cyclodextrins, hydroxypropyl beta-cyclodextrin, sulfobutylether beta-cyclodextrin, or a combination thereof. In certain embodiments, the carbohydrate is trehalose, sucrose, mannitol, sorbitol, or a combination thereof. Cyclodextrins are available in three different forms α, β, and γ based on the number of number of glucose monomers. The number of glucose monomers in α, β, and γ cyclodextrin can be 6, 7, or 8, respectively.

In some embodiments, the salt is sodium chloride, calcium chloride, potassium chloride, sodium hydroxide, stannous chloride, magnesium sulfate, sodium glucoheptonate, sodium pertechnetate, guanidine hydrochloride, potassium hydroxide, magnesium chloride, potassium nitrate, or a combination thereof. In certain embodiments, the salt is sodium chloride, sodium hydroxide, potassium nitrate, magnesium chloride, or a combination thereof.

In some embodiments, the chelator is disodium edetate, ethylenediaminetetraacetic acid, pentetic acid, or a combination thereof. In some embodiments, the mineral is calcium, zinc, titanium dioxide, or a combination thereof. In certain embodiments, the polymer is propyleneglycol, glucose star polymer, silicone polymer, polydimethylsiloxane, polyethylene glycol, carboxymethylcellulose, poly(glycolic acid), poly(lactic-co-glycolic acid), polylactic acid, polycaprolactone (PCL), polyvinylpyrrolidone (PVP), ficoll, dextran, or a combination thereof.

In some embodiments, the surfactant includes, but is not limited to: (i) cationic surfactants such as; cetyltrimethylammonium chloride, hexadecyltrimethylammonium chloride, benzalkonium chloride, benzethonium chloride, dioctadecyldimethylammonium bromide; (ii) anionic surfactants such as magnesium stearate, sodium dodecyl sulfate, dioctyl sodium sulfosuccinate, sodium myreth sulfate, perfluorooctanesulfonate, alkyl ether phosphates; (iii) non-ionic surfactants such as alkylphenol ethoxylates (TRITON™X-100), fatty alcohol ethoxylates (octaethylene glycol monododecyl ether, cocamide diethanolamine, poloxamers, glycerolmonostearate, fatty acid esters of sorbitol (sorbitan monolaurate, Tween 80, Tween 20, poloxamers (nonionic triblock copolymers composed of a central hydrophobic chain of polyoxypropylene (poly(propylene oxide)) flanked by two hydrophilic chains of polyoxyethylene (poly(ethylene oxide)) e.g., poloxamer 188, poloxamer 407; and (iv) zwitterionic surfactants such as cocamidopropyl hydroxysultaine, and 3-[(3-Cholamidopropyl)dimethylammonio]-1-propanesulfonate (CHAPS). In some embodiments, the surfactant is polysorbate, magnesium stearate, sodium dodecyl sulfate, alkylphenol ethoxylates, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan ester, phosphatidylcholine, polyglycerol polyricinoleate, siloxanes, cetyl polyethylene glycol/polypropylene glycol-10/1 dimethicone triglyceride, bis-polyethylene glycol/polypropylene glycol-14/14 dimethicone, bis-(glyceryl/lauryl) glyceryl lauryl dimethicone (&) caprylic/capric triglyceride, cetyl polyethylene glycol/polypropylene glycol-10/1 dimethicone, phospholipids, or a combination thereof. In certain embodiments, the surfactant is polysorbate, docusate, poloxamer or lecithin. In some embodiments, the surfactant is polysorbate 20, polysorbate 60, or polysorbate 80. In certain embodiments, the surfactant is polysorbate 20 or polysorbate 80, e.g., Tween 20, Tween 60, Tween 80. In some embodiments, the fatty acid ester of sorbitol is a sorbitan ester, e.g., span 20, span 40, span 60, or span 80. In certain embodiments, the surfactant is an ionic surfactant. In certain embodiments, the surfactant is polysorbate 80, polysorbate 20, or poloxamer 188. In some embodiments, the surfactant is polysorbate, magnesium stearate, sodium dodecyl sulfate, alkylphenol ethoxylates, glycerin, polyoxyethylated castor oil, docusate, sodium stearate, decyl glucoside, nonoxynol-9, cetyltrimethylammonium bromide, sodium bis(2-ethylhexyl) sulfosuccinate, sodium laureth sulfate, lecithin, sorbitan esters, or a combination thereof. In some embodiments, the surfactant is polysorbate, sorbitan esters, poloxamer 188, or a combination thereof.

In some embodiments, the protein stabilizer is acetyltryptophanate, caprylate, N-acetyltryptophan, trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, or a combination thereof. In some embodiments, the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, cyclodextrin, hydroxypropyl beta-cyclodextrin, sulfobutylether beta-cyclodextrin, or a combination thereof. In certain embodiments, the protein stabilizer is trehalose, cyclodextrin, hydroxypropyl beta-cyclodextrin, sulfobutylether beta-cyclodextrin, or a combination thereof. The stabilizers, used synonymously with the term “stabilizing agent”, as described herein, can be a salt, a carbohydrate, saccharides or amino acids, preferably a carbohydrate or saccharide admitted by the authorities as a suitable additive or excipient in pharmaceutical compositions. In certain embodiments, the PEG is PEG 200, PEG 300, PEG 3350, PEG 8000, PEG 10000, PEG 20000, or a combination thereof. The term “stabilizer” refers to an excipient or a mixture of excipients which stabilizes the physical and/or chemical properties of a protein, e.g., an antibody or antibody fragment. In some embodiments, stabilizers prevent, e.g., degradation of the protein during mixing, and/or storage of the particulate matter. Exemplary stabilizers include, but are not limited to, sugars, salts, hydrophobic salts, detergents, reducing agents, cyclodextrins, polyols, carboxylic acids, and amino acids. A “stable” formulation as described herein, refers to a particle formulation in which the protein retains an acceptable portion of its essential physical, chemical, or biological properties over an acceptable period of time. In the case of proteins, e.g., exemplary methods of assessing stability are reviewed in (i) Peptide and Protein Drug Delivery, 247-301, Vincent Lee Ed., Marcel Dekker, Inc., New York, NY, 1991, and (ii) Jones, A., Adv. Drug Delivery Rev. 10: 29-90 (1993). In certain embodiments, chemical stability of a protein is assessed by measuring the size distribution of the sample at several stages. These include, e.g., before particle formation (assessment of the aqueous feed solution), immediately after particle formation, and again after a period of storage, where storage takes place either within or in the absence of a suspension formulation carrier medium.

Examples of emulsifiers suitable for use in the particles include, but are not limited to, lipophilic agents having an HLB of less than 7, such as mixed fatty acid monoglycerides; mixed fatty acid diglycerides; mixtures of fatty acid mono- and diglycerides; lipophilic polyglycerol esters; glycerol esters including glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan ester including sorbitan monostearate, sorbitan sesquioleate; fatty acids and their soaps including stearic acid, palmitic acid, and oleic acid; and mixtures thereof glyceryl monooleate, glyceryl dioleate, glyceryl monostearate, glyceryl distearate, glyceryl monopalmitate, and glyceryl dipalmitate; glyceryl-lacto esters of fatty acids; propylene glycol esters including propylene glycol monopalmitate, propylene glycol monostearate, and propylene glycol monooleate; sorbitan ester including sorbitan monostearate, sorbitan sesquioleate; fatty acids and their soaps including stearic acid, palmitic acid, and oleic acid; phospholipids; or a combination thereof. In some embodiments, the emulsifier is polysorbate 80, polysorbate 60, polysorbate 20, e.g., Tween 80, Tween 60, Tween 20, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof. In some embodiments, the fatty acid ester of sorbitol is a sorbitan ester, e.g., span 20, span 40, span 60, or span 80. In certain embodiments, the emulsifier is polysorbate 80, sorbitan monooleate, or a combination thereof.

In some embodiments, the antiseptic is phenol, m-cresol, benzyl alcohol, 2-phenyloxyethanol, chlorobutanol, neomycin, benzethonium chloride, gluteraldehyde, beta-propiolactone, or a combination thereof.

In some embodiments, the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or various salts thereof (arginine hydrochloride, arginine glutamate, or the like) or a combination thereof. In some embodiments, the amino acid is alanine, aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glycine, glutamine, proline, or a combination thereof. In some embodiments, the amino acid is arginine, histidine, proline, asparagine, or a combination thereof. In certain embodiments, the amino acid is histidine.

In some embodiments, the antioxidant is glutathione, ascorbic acid, cysteine, N-acety-L-tryptophanate, tocopherol, histidine, methionine, pentetic acid, or a combination thereof. In some embodiments, the organic solvent is dimethyl sulfoxide, N-methyl-2-pyrrolidone, or a combination thereof. The paraben is a parahydroxybenzoate. In some embodiments, the bactericide is benzalkonium chloride (cationic surfactants), hypochlorites, peroxides, alcohols, phenolic compounds (e.g. carbolic acid), benzyl benzoate, or a combination thereof. In certain embodiments, the bactericide is benzalkonium chloride or benzyl benzoate.

In some embodiments, the preservative is sodium nitrate, sulfur dioxide, potassium sorbate, sodium sorbate, sodium benzoate, benzoic acid, methyl hydroxybenzoate, thimerosal, parabens, formaldehyde, castor oil, or a combination thereof. In certain embodiments, the preservative is methyl hydroxybenzoate, thimerosal, a paraben, formaldehyde, castor oil, or a combination thereof. In some embodiments, the analgesic is acetaminophen or lidocaine.

In some embodiments, the particles have a pH adjusting agent and excipient content of less than about 30% by mass, e.g., less than about 29, 25, 20, 15, 10, 9, 8, 7, 6, 5, 4, 3, 2, 1% by mass. In some embodiments, the particles have a pH adjusting agent and excipient content of less than about 20% by mass. In certain embodiments, the particles have a pH adjusting agent and excipient content of less than about 15% by mass. In some embodiments, the particles have a pH adjusting agent and excipient content of less than about 10% by mass. In certain embodiments, the particles have a pH adjusting agent and excipient content of less than about 5% by mass.

As used herein, the terms “moisture” and “water” may be used interchangeably. In some embodiments, the residual first aqueous liquid, e.g., water, and/or organic liquid content remaining in the particles is less than about 10% by mass after removing the first aqueous liquid and organic liquid, e.g., less than about 9, 8, 7, 6, 5, 4, 3, 2, 1, or 0.5% by mass after removing the first aqueous liquid and organic liquid. In some embodiments, the residual first aqueous liquid content remaining in the particles is less than about 10% by mass after removing the first aqueous liquid and organic liquid, e.g., less than about 9, 8, 7, 6, 5, 4, 3, 2, 1, or 0.5% by mass after removing the first aqueous liquid and organic liquid. In certain embodiments, the residual first aqueous liquid content remaining in the particles is less than about 5% by mass after removing the first aqueous liquid and organic liquid. In some embodiments, the residual first aqueous liquid content remaining in the particles is less than about 3% by mass after removing the first aqueous liquid and organic liquid. In certain embodiments, the residual first aqueous liquid content remaining in the particles is about 1% to about 3% by mass after removing the first aqueous liquid and organic liquid. In some embodiments, the residual organic liquid content remaining in the particles is less than about 5% by mass after removing the first aqueous liquid and organic liquid, e.g., less than about 4, 3, 2, 1, or 0.5% by mass after removing the first aqueous liquid and organic liquid.

Exemplary methods for the measurement of residual first aqueous liquid and organic liquid content include chemical titration methods, e.g., Karl Fischer titration involving an oven. A variety of solvents, including water, may also be measured using weight loss methods involving thermal excitation. Exemplary methods include Thermogravimetric Analysis with Infrared Spectrometry (TGA-IR) or Gas Chromatography Flame Ionization Detector Mass Spectrometry (GC-FID/MS).

As used herein, the term “dispersity index” (DI) is a parameter characterizing the degree of non-uniformity of a size distribution of particles. The polydispersity index (PDI), “population dispersity” or “span”, e.g., D10, D50, D90, can also mean a value that indicates the breadth of the particle size distribution. Particle size distribution are reported by D10, D50, D90, and the mean particle size in μm, with the values representing the percentage of particles that are smaller than the indicated D-number, e.g. the D10 particle size is the particle diameter at which 10% of the mass is composed of particles with a diameter less than this value, the D50 particle size is the particle diameter at which 50% of the mass is composed of particles with a diameter less than this value and the D90 particle size is the particle diameter at which 90% of the mass is composed of particles with a diameter less than this value. The D10, D50, and D90 particle size distribution can be measured using a laser light scattering particle sizer.

In some embodiments, the particles have less than about 5% aggregation of the protein. In some embodiments, the particles have less than about 3% aggregation of the protein. In certain embodiments, the particles have less than about 1% aggregation of the protein. In some embodiments, the particles have less than about 0.5% aggregation of the protein. In some embodiments, the particles are substantially free from any aggregation of the protein. In certain embodiments, the particles have less than about 5% aggregation of the protein after processing.

In some embodiments, the particles have less than about 5% fragmentation of the protein. In some embodiments, the particles have less than about 3% fragmentation of the protein. In certain embodiments, the particles have less than about 1% fragmentation of the protein. In some embodiments, the particles are substantially free from any fragmentation of the protein. In certain embodiments, the particles have less than about 5% fragmentation of the protein after processing. Suitable methods for measuring aggregation and fragmentation of a protein can be accomplished by using size-exclusion chromatography (SEC).

In some embodiments, the method provides less than about 5% change in charge variants in the population of a protein, e.g., an antibody or an antibody fragment, (e.g., less than about 4, 3, 2, or 1%) as compared to the protein prior to particle formation. In certain embodiments, the particles have less than about 5% change in charge variants in the population of a protein after processing. Charge variants may be acidic, basic, or neutral, and the variation may be caused post-translation modifications at terminal amino acids, such as asparagine deamidation or lysine glycation. For example, charge variants include the loss of a positive charge by the loss of a C-terminal lysine residue, covalent bonding of the amine portions of two lysine residues by reducing sugars, or the conversion of an N-terminal amine to a neutral amide by the cyclization of N-terminal glutamines. Negative charges on proteins, e.g., antibodies, can appear by the conversion of asparagine residues to aspartic acid and/or isoaspartic residues via a deamidation reaction.

Exemplary methods of measuring charge variants include cation exchange chromatography (CIEX), where the variants are quantified by dividing the area under the peak corresponding to the variant, e.g., acidic, basic, or neutral population by the cumulative area contained beneath all peaks in the sample spectrum. Changes in charge variant population percentage between two samples, e.g., Sample A and Sample B, are computed as the numerical difference in the respective population variant percentages, i.e., by subtracting the specific variant, e.g., acidic, percentage of Sample B from the specific variant, e.g., acidic, percentage of Sample A, or vice versa. In some embodiments, the analysis may be extended similarly for all variants within a population.

In some embodiments, the particles have less than about 5% change in charge variants of the protein. In some embodiments, the particles have less than about 3% change in charge variants of the protein. In certain embodiments, the particles have less than about 1% change in charge variants of the protein. In some embodiments, the particles are substantially free from any change in charge variants of the protein. Suitable methods for measuring a change in charge variants of a protein can be accomplished by using cation exchange chromatography (CIEX).

The particles comprising at least one protein described herein, can be characterized in a number of ways, as well as any methods of characterizing the particles disclosed in, for example, in International Application Nos. PCT/US2017/063150 (Pub. No. WO 2018/098376), PCT/US2018/043774 (Pub. No. WO 2019/023392), PCT/US2019/033875 (Pub. No. WO 2019/226969), PCT/US2020/15957 (Pub. No. WO 2020/160323), PCT/US2020/050508 (Pub. No. WO 2021/050953), PCT/US2021/16878, PCT/US2021/018806, and PCT/US2021/027755, the contents of each of which are hereby incorporated by reference in their entireties.

While each of the elements of the present disclosure is described herein, as containing multiple embodiments, it should be understood that, unless indicated otherwise, each of the embodiments of a given element of the present disclosure is capable of being used with each of the embodiments of the other elements of the present disclosure and each such use is intended to form a distinct embodiment of the present disclosure.

It will be understood by one of ordinary skill in the relevant arts that other suitable modifications and adaptations to the methods described herein are readily apparent from the description of the disclosure contained herein, in view of information known to the ordinarily skilled artisan, and may be made without departing from the scope of the disclosure or any embodiment thereof.

### Methods of the Disclosure

The methods described herein, are generally provided for screening formulations comprising particles for stability of a protein in the particles, comprising the steps of: a) dispensing a plurality of different mixtures into a plurality of wells, each mixture comprising a quantity of particles, a first aqueous liquid, and an organic liquid, wherein the particles comprise a protein and one or more pH adjusting agents, and the circularity of the particles is from about 0.80 to about 1.00; b) removing the first aqueous liquid and the organic liquid from each well to provide a formulation comprising particles in each well; c) dissolving the particles in a second aqueous liquid and determining the stability of the protein in each well after the first aqueous liquid and the organic liquid have been removed; and d) selecting a formulation based on the stability of the protein determined in c). In some embodiments, the mixture comprising a quantity of particles, a first aqueous liquid and an organic liquid in a) is prepared by: i. adding a quantity of a protein in a first aqueous liquid into containers to provide a plurality of containers, each container comprising a quantity of the protein in the first aqueous liquid; ii. adding one or more pH adjusting agents to each container; iii. adding an organic liquid to each container; and iv. mixing the first aqueous liquid comprising the protein and the pH adjusting agent with the organic liquid in each container, thereby preparing the mixture comprising a quantity of particles, a first aqueous liquid, and an organic liquid in each container, wherein the particles comprise the protein and one or more pH adjusting agents. In some embodiments, the protein is a therapeutic biologic. In certain embodiments, the therapeutic biologic is an antibody or fragment thereof. In some embodiments, the protein is bovine serum albumin (BSA) or human serum albumin (HSA). In some embodiments, the protein, e.g., therapeutic biologic, has an activity per unit of about 0.8 to about 1.0. In certain embodiments, the circularity of the particles in each well is from about 0.80 to about 1.00.

The methods as disclosed herein, allow not only for the high-throughput selection of suitable particle formulations of proteins but also for predicting stability properties under manufacturing and long-term storage conditions. In some embodiments, the methods, e.g., high throughput screening techniques, are used in combination with accelerated stability studies to test the impact of particle formulation parameters such as PH levels, buffering species, excipients, surfactants, and salts on protein stability. In some embodiments, the studies are conducted under suboptimal conditions, e.g., elevated temperatures, in order to facilitate protein degradation in short periods of time. As disclosed herein, accelerated stability studies at high temperature, e.g., 40-60° C., allow for the assessment of the routes of protein degradation in short periods of time and creates the opportunity to streamline protein particle formulation development starting from early pre-formulation screening to commercial formulation development. Specifically, accelerated stability of lead particle formulation candidates can predict and identify a development protein particle formulation based on the design of the methods described herein. In certain embodiments, the disclosure provides a method, e.g., high throughput method, for screening particle formulations for protein stability.

In some embodiments, the methods comprise dispensing a quantity of a protein, e.g., therapeutic biologic, in a first aqueous liquid into containers to provide a plurality of containers, each container comprising a quantity of the protein in the first aqueous liquid. In some embodiments, the quantity of a protein in a first aqueous liquid that is added to a container is from about 10 uL to about 50 uL. In certain embodiments, the quantity of a protein in a first aqueous liquid added to a container is from about 20 uL to about 200 uL.

The term “viscosity” is used to describe the property of a fluid acting to resist shearing flow. For the purposes of the present disclosure, viscosity can be determined using a rheometer, e.g., AR-G2 Rheometer (TA Instruments, USA), fitted with a cone and plate (2°/40 mm) at 25° C. at a specified shear rate. In some embodiments, the viscosity is measured at a shear rate in the Newtonian regime. In some embodiments, the viscosity is measured at a shear rate of 100 s⁻¹ or greater, e.g., at 1000 s⁻¹ or greater than 1000 s⁻¹, or greater than 10,000 s⁻¹ or greater than 50,000 s⁻¹. The term “low viscosity” as used herein, describes a composition, e.g., a liquid carrier, having a viscosity of less than about 200 mPa·s.

In some embodiments, the first aqueous liquid comprising the protein and pH adjusting agent, optionally, and one or more excipients, has a viscosity of less than about 200 mPa·s, less than about 150 mPa·s, less than about 125 mPa·s, less than about 100 mPa·s, less than about 95 mPa·s, less than about 90 mPa·s, less than about 85 mPa·s, less than about 80 mPa·s, less than about 75 mPa·s, less than about 70 mPa·s, less than about 65 mPa·s, less than about 60 mPa·s, less than about 55 mPa·s, less than about 50 mPa·s, less than about 45 mPa·s, less than about 40 mPa·s, less than about 35 mPa·s, less than about 30 mPa·s, less than about 25 mPa·s, less than about 20 mPa·s, less than about 19 mPa·s, less than about 18 mPa·s, less than about 17 mPa·s, less than about 16 mPa·s, less than about 15 mPa·s, less than about 14 mPa·s, less than about 13 mPa·s, less than about 12 mPa·s, less than about 11 mPa·s, less than about 10 mPa·s, less than about 9.5 mPa·s, less than about 9 mPa·s, less than about 8.5 mPa·s, less than about 8 mPa·s, less than about 7.5 mPa·s, less than about 7 mPa·s, less than about 6.5 mPa·s, less than about 6 mPa·s, less than about 5.5 mPa·s, less than about 5 mPa·s, less than about 4.5 mPa·s, less than about 4 mPa·s, less than about 3.5 mPa·s, less than about 3 mPa·s, less than about 2.5 mPa·s, less than about 2 mPa·s, less than about 1.5 mPa·s, less than about 1 mPa·s, less than about 0.5 mPa·s, less than about 0.1 mPa·s, less than about 0.05 mPa·s, or less than about 0.01 mPa·s (one millipascal-second). In some embodiments, the first aqueous liquid comprising the protein and pH adjusting agent, optionally, and one or more excipients, has a viscosity of about 0.01 mPa·s to about 10,000 mPa·s, e.g., about 0.01 mPa·s to about 1,000 mPa·s, about 0.01 mPa·s to about 100 mPa·s, about 0.01 mPa·s to about 50 mPa·s, about 0.01 mPa·s to about 25 mPa·s, about 0.01 mPa·s to about 10 mPa·s, about 0.01 mPa·s to about 5 mPa·s, or about 0.01 mPa·s to about 1 mPa·s. In some embodiments, the viscosity of the first aqueous liquid comprising the protein and pH adjusting agent, optionally, and one or more excipients, can range of about 0.27 mPa·s to about 200 mPa·s, e.g., about 0.27 mPa·s to about 50 mPa·s, about 1 mPa·s to about 30 mPa·s, or about 20 mPa·s to about 50 mPa·s. In some embodiments, the viscosity of the first aqueous liquid comprising the protein and pH adjusting agent, optionally, and one or more excipients, ranges of about 0.27 mPa·s to about 200 mPa·s, e.g., about 0.27 mPa·s to about 100 mPa·s, about 0.27 mPa·s to about 50 mPa·s, about 0.27 mPa·s to about 30 mPa·s, about 1 mPa·s to about 20 mPa·s, or about 1 mPa·s to about 15 mPa·s. In certain embodiments, the viscosity is measured at a shear rate in the Newtonian regime. In some embodiments, the viscosity is measured at a shear rate of about 100 s⁻¹ or greater, e.g., at about 1000 s⁻¹ or greater than about 1000 s⁻¹. Methods of controlling viscosity include temperature regulation and viscosity modifying additives. In some embodiments, mixtures of liquids may also be used to control viscosity. The units “mPa·s” and “cP” are used herein interchangeably in the broadest sense.

In some embodiments, the first aqueous liquid comprising the protein and pH adjusting agent, optionally, and one or more excipients, has a viscosity of less than about 200 mPa·s. In some embodiments, the first aqueous liquid comprising the protein and pH adjusting agent, optionally, and one or more excipients, has a viscosity of less than about 150 mPa·s. In some embodiments, the first aqueous liquid comprising the protein and pH adjusting agent, optionally, and one or more excipients, has a viscosity of less than about 100 mPa·s. In some embodiments, the first aqueous liquid comprising the protein and pH adjusting agent, optionally, and one or more excipients, has a viscosity of less than about 80 mPa·s. In some embodiments, the first aqueous liquid comprising the protein and pH adjusting agent, optionally, and one or more excipients, has a viscosity of less than about 50 mPa·s. In some embodiments, the first aqueous liquid comprising the protein and pH adjusting agent, optionally, and one or more excipients, has a viscosity of less than about 40 mPa·s. In some embodiments, the first aqueous liquid comprising the protein and pH adjusting agent, optionally, and one or more excipients, has a viscosity of less than about 30 mPa·s. In some embodiments, the first aqueous liquid comprising the protein and pH adjusting agent, optionally, and one or more excipients, has a viscosity of less than about 20 mPa·s. In some embodiments, the first aqueous liquid comprising the protein and pH adjusting agent, optionally, and one or more excipients, has a viscosity of less than about 10 mPa·s. In some embodiments, the first aqueous liquid comprising the protein and pH adjusting agent, optionally, and one or more excipients, has a viscosity of less than about 5 mPa·s. In some embodiments, the first aqueous liquid comprising the protein and pH adjusting agent, optionally, and one or more excipients, has a viscosity of less than about 3 mPa·s. In some embodiments, the first aqueous liquid comprising the protein and pH adjusting agent, optionally, and one or more excipients, has a viscosity of less than about 2.5 mPa·s.

In some embodiments, the first aqueous liquid is water, 0.9% saline, lactated Ringer's solution, buffers, dextrose 5%, or a combination thereof. In certain embodiments, the first aqueous liquid is water. Exemplary buffers of the disclosure may include acetate buffer, histidine buffer, succinate buffer, HEPES buffer, tris buffer, carbonate buffer, citrate buffer, phosphate buffer, phosphate-buffered saline, glycine buffer, barbital buffer, cacodylate buffer, ammonium formate buffer, urea solution, or a combination thereof.

“Container” or “sample container” may consist of any suitable material, such as glass, organic polymers, such as polycarbonate, polystyrene, or the like, ceramic, metal or any other material typically employed to hold reagents. Other examples of suitable containers include tubes that may be fabricated from similar substances as centrifuge tubes, that may consist of lined interiors. Other containers include tubes, vials, ampules, envelopes, flasks, bottles, or the like. In some embodiments, the container is a centrifuge tube. Containers may have a sterile access port, such as a tube having a stopper or lid. Other containers may have two compartments that are separated by a readily removable membrane that upon removal permits the components to mix e.g., a protein in a first aqueous liquid in one compartment, and a dehydrating organic liquid in the other. Removable membranes may be glass, plastic, rubber, or the like. In certain embodiments, the container is in the form of a plurality of containers to provide a plurality of containers each comprising a quantity of the protein in a first aqueous liquid. The containers are typically dispensed with sample liquids by a user either manually or by automated liquid handling. Typically, the container or a plurality of containers are all sealed by placing a foil or parafilm over the container or a plurality of containers which prevents the sample liquids from flowing out of the respective containers in order to avoid contamination or concentration mismatches due to evaporation. In certain embodiments, the container is a closed container with a tapered bottom that can be inverted and/or vortexed to promote optimal mixing. In some embodiments, the containers comprise tubes, vials, ampules, envelopes, flasks, or bottles.

In some embodiments, the method in step ii. adds one or more pH adjusting agents to each container. In certain embodiments, the pH adjusting agent is dispensed manually or by automated liquid handling.

In some embodiments, the organic liquid is acetonitrile, chlorobenzene, chloroform, cyclohexane, cumene, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, methylisobutylketone, N-methylpyrrolidone, nitromethane, pyridine, sulfolane, tetrahydrofuran, tetralin, toluene, 1,1,2-trichloroethene, xylene, acetic acid, acetone, anisole, 1-butanol, 2-butanol, butylacetate, tert-butylmethyl ether, dimethyl sulfoxide, ethanol, ethylacetate, ethyl ether, ethyl formate, formic acid, heptane, isobutylacetate, isopropylacetate, methylacetate, 3-methyl-1-butanol, methylethyl ketone, 2-methyl-1-propanol, pentane, 1-pentanol, 1-propanol, 2-propanol, propylacetate, triethylamine, 1,1-diethoxypropane, 1,1-dimethoxymethane, 2,2-dimethoxypropane, isooctane, isopropyl ether, methylisopropyl ketone, methyltetrahydrofuran, petroleum ether, trichloroacetic acid, trifluoroacetic acid, decanol, 2-ethylhexylacetate, amylacetate, or a combination thereof. In certain embodiments, the organic liquid is ethyl acetate or butylacetate. In some embodiments, the method in step iii. selects an organic liquid and is added to each container. In certain embodiments, the organic liquid is dispensed manually or by automated liquid handling.

In some embodiments, step ii. further comprises adding one or more different excipients to each container. In some embodiments, the one or more different excipients are dispensed manually or by automated liquid handling. In certain embodiments, the one or more different excipients is selected from one or more of a carbohydrate, a salt, a chelator, a mineral, a polymer, a surfactant, a protein stabilizer, an emulsifier, an antiseptic, an amino acid, an antioxidant, an organic solvent, a paraben, a bactericide, a preservative, and an analgesic. In some embodiments, the excipient is tryptophan, hydroxypropyl beta-cyclodextrin, isoleucine, glycine, threonine, glutamine, asparagine, or a combination thereof. In some embodiments, the excipient further comprises at least one pharmaceutically acceptable additive, diluent, carrier, or a combination thereof.

In some embodiments, the method comprises mixing the first aqueous liquid comprising the protein and pH adjusting agent with the organic liquid, thereby forming particles comprising the protein and pH adjusting agent in each container, wherein the circularity of the particles in each well is from about 0.80 to about 1.00. In some embodiments, the method in step iv. comprises mixing the first aqueous liquid comprising the protein and pH adjusting agent with the organic liquid, thereby forming particles comprising the protein and pH adjusting agent in each container, wherein the circularity of the particles in each well is from about 0.80 to about 1.00. In certain embodiments, the method comprises mixing the first aqueous liquid comprising the protein, pH adjusting agent and one or more excipients with the organic liquid, thereby forming particles comprising the protein and pH adjusting agent in each container, wherein the circularity of the particles in each well is from about 0.80 to about 1.00. In some embodiments, the method in step iv. comprises mixing the first aqueous liquid comprising the protein, pH adjusting agent and one or more excipients with the organic liquid, thereby forming particles comprising the protein, pH adjusting agent and one or more excipients in each container, wherein the circularity of the particles in each well is from about 0.80 to about 1.00.

In some embodiments, the particles of the disclosure are formed by mixing the first aqueous liquid comprising the protein and pH adjusting agent with the organic liquid. In some embodiments, the particles are formed by mixing the first aqueous liquid comprising the protein, pH adjusting agent and one or more excipients with the organic liquid. In some embodiments, the mixing comprises mechanical stirring, mechanical shaking, mechanical stirring, vortexing, or sonication. In certain embodiments, the particles of the disclosure are formed by vortexing or sonication to promote optimal mixing.

In some embodiments, the circularity of the particles is about 0.80 to about 1.00, e.g., about 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99 to about 1.00. In some embodiments, the particles have a circularity of about 0.85 to about 1.00. In some embodiments, the particles have a circularity of about 0.90 to about 1.00. In some embodiments, the particles have a circularity of about 0.95 to about 1.00. In certain embodiments, the particles have a circularity of about 0.98 to about 1.00. In some embodiments, the particles have a circularity of about 1.00. Methods of measuring particle circularity include image analysis of scanning electron micrographs of the particles in which the average roundness is calculated on the basis of the cross-sectional shapes of the particles projected onto the plane of the image. Such roundness factors can be extended to identify the corresponding circularity.

In some embodiments, the particles have less than about 10% internal void spaces. In some embodiments, the particles have less than about 5% internal void spaces. In certain embodiments, the particles have less than about 3% internal void spaces. In some embodiments, the particles have less than about 1% internal void spaces. In certain embodiments, the particles are substantially free from any internal void spaces.

In some embodiments, the particles have greater than about 60% protein by weight. In some embodiments, the particles have greater than about 70% protein by weight. In some embodiments, the particles have greater than about 80% protein by weight. In certain embodiments, the particles have greater than about 90% protein by weight. In some embodiments, the particles have greater than about 95% protein by weight. In some embodiments, the particles have greater than about 98% protein by weight.

In some embodiments, the method comprises dispensing a plurality of different mixtures into a plurality of wells, each mixture comprising a quantity of particles, a first aqueous liquid, and an organic liquid. In some embodiments, the method comprises dispensing a plurality of different mixtures into a plurality of wells, each mixture comprising a quantity of particles, a first aqueous liquid, and an organic liquid. A quantity of particles, a first aqueous liquid and an organic liquid are typically dispensed from each container into each well of a plurality of wells by a user either manually or by automated liquid handling.

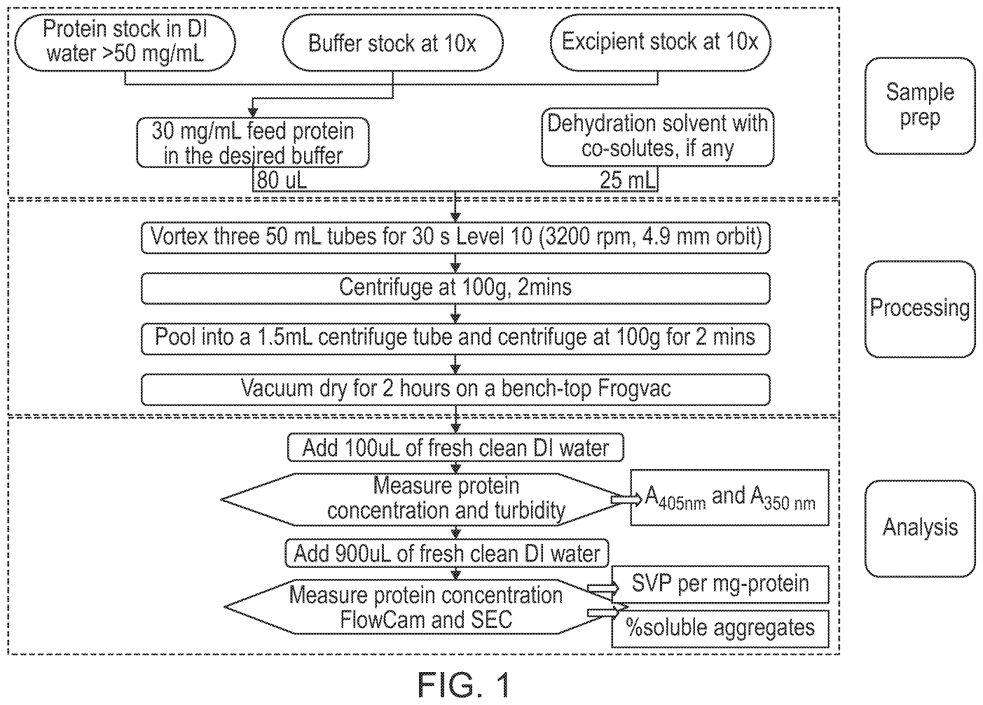

A plurality of wells, each of which can hold a sample, e.g., a quantity of particles, a first aqueous liquid, an organic liquid, and optionally, one or more different excipients is disclosed herein. The plurality of wells is typically in the form of a micro well plate, such as industry standard 96-well or 384-well micro titer plates, or in the form of a plurality of vials. The micro well plates are filled with sample by a user either manually or by automated liquid handling. Typically, the wells of the micro well plate are all sealed by placing a foil or parafilm over the micro well plate which prevents the samples from flowing out of the respective wells in order to avoid contamination or concentration mismatches due to evaporation. In the exemplary embodiment in Example 1, FIG. 1, the micro well plate comprises 96 wells, e.g. is a 96-well micro titer plate containing twelve rows of eight wells each, with a feed amount of 80 uL, centrifuge speed at 100 g, vacuum drying for 2 hours. Alternatively, a feed amount of 120 uL, centrifuge speed at 500 g, vacuum drying for 12 hours can be used.

As disclosed herein, the first aqueous liquid and organic liquid is removed from each well. In some embodiments, the method comprises removing the first aqueous liquid and organic liquid from each well to provide a formulation comprising particles in each well. The term “secondary desiccation” refers to a post-processing step, e.g., after removal of the first aqueous liquid and organic liquid by which the residual moisture and/or organic liquid content of the particles is modified. Exemplary methods of secondary desiccation include vacuum drying, with or without the application of heat, lyophilization, fluidized bed drying, tray drying, or belt drying. Secondary desiccation may also be used to remove any washing liquids that are used to separate the particles from the first aqueous liquid and organic liquid. In some embodiments, the first aqueous liquid and organic liquid are removed through centrifugation, sieving, filtration, magnetic collection, solvent exchange, or decanting. In certain embodiments, the first aqueous liquid and organic liquid is removed by evaporation, vacuum desiccation or lyophilization, e.g., vacuum drying, with or without the application of heat, lyophilization, fluidized bed drying, tray drying, or belt drying. In some embodiments, the first aqueous liquid and organic liquid is removed by lyophilization or vacuum desiccation. In certain embodiments, the first aqueous liquid and organic liquid is removed by lyophilization or vacuum desiccation.

In some embodiments, the residual moisture and/or liquid content of the particles is less than about 7% by weight, e.g., less than about 6, 5, 4, 3, 2, 1.9, 1.8, 1.7, 1.6, 1.5, 1.4, 1.3, 1.2, 1.1, 1, 0.9, 0.8, 0.7, 0.6, 0.5, 0.4, 0.3, 0.2, or 0.1% by weight. In some embodiments, the particles have less than about 7% residual moisture by weight. In some embodiments, the particles have less than about 5% residual moisture by weight. In certain embodiments, the particles have less than about 3% residual moisture by weight. In some embodiments, the particles have less than about 1% residual moisture by weight.

Exemplary methods for the measurement of moisture content include chemical titration methods, e.g., Karl Fischer titration involving an oven. A variety of solvents, including water, may also be measured using weight loss methods involving thermal excitation. Exemplary methods include Thermogravimetric Analysis with Infrared Spectroscopy (TGA-IR) or Gas Chromatography Flame Ionization Detector Gas Chromatography (GC-FID/MS).

In certain embodiments according to the disclosure, the methods as described herein, allows the determination of stable particle formulations having improved stability of the protein compared to a composition comprising the protein in the first aqueous liquid. A “stable” formulation is one in which all the protein therein essentially retains their physical stability and/or chemical stability and/or biological activity upon storage at the intended storage temperature, e.g. 4-60° C. It is desired that the particle formulation essentially retains its physical and chemical stability, as well as its biological activity upon storage. The storage period is generally selected based on the intended shelf-life of the particle formulation. Furthermore, the particle formulation should be stable following freezing (to, e.g., −70° C.) and thawing of the particle formulation, for example following 1, 2 or 3 cycles of freezing and thawing. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography or capillary zone electrophoresis; amino-terminal or carboxy-terminal sequence analysis; mass spectrometric analysis; SDS-PAGE analysis to compare reduced and intact antibody (fragmentation); peptide map (for example tryptic or LYS-C) analysis; evaluating biological activity or antigen binding function of the antibody; or the like.

In some embodiments, the methods as disclosed herein, relate to determining the stability of the protein in each well. In certain embodiments, the stability of the protein is determined by measuring aggregation, fragmentation, change in charge variants, Subvisible Particles (SvPs), turbidity, or a combination thereof, of the protein.

In some embodiments, the particles have less than about 5% aggregation of the protein. In some embodiments, the particles have less than about 3% aggregation of the protein. In certain embodiments, the particles have less than about 1% aggregation of the protein. In some embodiments, the particles have less than about 0.5% aggregation of the protein. In certain embodiments, the particles are substantially free from any aggregation of the protein.

In some embodiments, the particles have less than about 5% fragmentation of the protein. In some embodiments, the particles have less than about 3% fragmentation of the protein. In certain embodiments, the particles have less than about 1% fragmentation of the protein. In some embodiments, the particles are substantially free from any fragmentation of the protein.

In some embodiments, the particles have less than about 5% change in charge variants of the protein. In some embodiments, the particles have less than about 3% change in charge variants of the protein. In certain embodiments, the particles have less than about 1% change in charge variants of the protein. In some embodiments, the particles are substantially free from any change in charge variants of the protein.

In some embodiments, insoluble particulate matter with characteristic sizes greater than or equal to about 100 μm that persist upon dissolution in a second aqueous liquid are referred to as Visible Particles (VP). In certain embodiments of the disclosure described herein, the particles are substantially free of Visible Particles (VP). In some embodiments, the second aqueous liquid is water, aqueous buffer, e.g., phosphate buffer or phosphate-buffered saline (PBS), or a physiologically relevant aqueous liquid. In certain embodiments, the aqueous buffer is water, phosphate buffer, or phosphate-buffered saline (PBS). As used herein, the term “physiologically relevant aqueous liquid” refers to any water-containing body fluid distributed in the extracellular compartment, e.g., extracellular fluid, interstitial fluid, intravascular fluid (blood, plasma, and lymph) and cerebrospinal fluid. In some embodiments, the particles are substantially free of Visible Particles (VP) upon dissolution in a second aqueous liquid. In certain embodiments of the disclosure, the particles described herein, immediately dissolves upon dissolution in a second aqueous liquid.

In some embodiments, insoluble particulate matter with characteristic sizes of about 1 μm to about 100 μm that persist upon dissolution in a second aqueous liquid are referred to as Subvisible Particles (SvPs). SvPs are present in quantities of about 0 to 100,000,000 per mL, e.g., about 0 to about 10,000,000 per mL, about 0 to about 1,000,000 per mL, about 0 to about 500,000 per mL, about 0 to about 100,000 per mL, about 0 to about 50,000 per mL, about 0 to about 10,000 per mL, about 0 to about 6,000 per mL, about 0 to about 1,000 per mL, about 0 to about 600 per mL, about 0 to about 250 per mL, about 0 to about 100 per mL, about 0 to about 60 per mL, or about 0 to about 10 per mL. In some embodiments, the count of particles with characteristic size greater than or equal to 10 μm is about 0 to about 6,000 per mL, e.g., about 0 to about 1,000 per mL, about 0 to about 100 per mL, about 0 to about 10 per mL, about 0 to about 5 per mL, about 0 to about 3 per mL, or about 0 to about 1 per mL. In certain embodiments, the count of particles with characteristic size greater than or equal to 25 μm is about 0 to about 600 per mL, e.g., about 0 to about 100 per mL, about 0 to about 10 per mL, about 0 to about 3 per mL, about 0 to about 1 per mL, about 0 to about 0.5 per mL, or about 0 to about 0.1 per mL. Exemplary methods of measuring SvPs include analysis of the protein with a Coulter Counter, HIAC Royco, or micro-flow imaging system after reconstitution and dilution of the protein to a standard concentration, e.g., about 100 mg/mL or about 1 mg/mL. In some embodiments, the particles have a concentration of insoluble Subvisible Particles (SvPs) having a characteristic size of greater than about 10 μm of about 0 per mL to about 100,000,000 per mL upon dissolution in a second aqueous liquid. In some embodiments, the particles have a concentration of insoluble Subvisible Particles (SvPs) having a characteristic size of greater than about 10 μm of about 0 per mL to about 6000 per mL upon dissolution in a second aqueous liquid. In certain embodiments, the particles have a concentration of insoluble Subvisible Particles (SvPs) having a characteristic size of greater than about 25 μm of about 0 per mL to about 600 per mL upon dissolution in a second aqueous liquid. In some embodiments, the particles are substantially free of insoluble Subvisible Particles (SvPs) upon dissolution in a second aqueous liquid.

In some embodiments, insoluble particulate matter with characteristic sizes of about 100 nm to about 1 μm that persist upon dissolution in a second aqueous liquid are referred to as submicron particles (SMP) and sometimes known as nanoparticles. The presence of such SMPs is thought to contribute to immunogenicity and thus should be avoided to minimize such effects. Quantitatively, SMPs are present in quantities of about 0 to 5×1012 per mL, e.g., about 0 to about 0.5×1012 per mL, about 0 to about 50×10⁹ per mL, about 0 to about 10×10⁹ per mL, about 0 to about 5×10⁹ per mL, about 0 to about 0.5×10⁹ per mL, about 0 to about 50×10⁶ per mL, about 0 to about 1×10⁶ per mL, about 0 to about 500,000 per mL, about 0 to about 200,000 per mL, about 0 to about 100,000 per mL, about 0 to about 10,000 per mL, about 0 to about 5000 per mL, or about 0 to about 1000 per mL. Exemplary methods of measuring SMPs quantitatively include analysis of the protein with a NanoSight, asymmetric field flow fractionation coupled to a multi-angle laser light scattering (AF4 MALS), Dynamic Light Scattering (DLS), or any other nano-particle tracking device known in the art after reconstitution in a second aqueous liquid and dilution of the protein to a standard concentration, e.g., about 100 mg/mL, about 1 mg/mL, or about 1 μg/mL. Qualitatively, SMPs are within a range comparable to the starting protein in a first aqueous liquid. In certain embodiments, the composition is substantially free of submicron particles (SMP) upon dissolution in a second aqueous liquid.

Turbidity is an optical property of fluids, and is proportional to the amount of insoluble particulate matter that is suspended in the fluid to scatter light passing through the fluid. In certain embodiments, the particles upon dissolution in a second aqueous liquid have a substantially similar turbidity compared to a composition comprising the protein in the first aqueous liquid. In certain embodiments, the particles upon dissolution in a second aqueous liquid are substantially free of turbidity.

As disclosed herein, the term “immunogenicity” refers to the induction of an immune response by an injected composition of the protein (the antigen), while “antigenicity” refers to the reaction of the protein particle formulation with preexisting antibodies. Collectively, antigenicity and immunogenicity are referred to as “immunoreactivity”. In certain embodiments, the particles have substantially similar immunogenicity compared to a composition comprising the protein in the first aqueous liquid. In certain embodiments, the particles are substantially non-immunogenic.

In some embodiments of the disclosure, the particle formulations have improved stability of the protein compared to a composition comprising the protein in the first aqueous liquid. In certain embodiments, the particles have improved stability of the protein compared to a composition comprising the protein in the first aqueous liquid.

In some embodiments, the particles are stable for at least one month. In some embodiments, the particles are stable for at least two months. In some embodiments, the particles are stable for at least two months at 40° C. In some embodiments, the particles are stable for at least three months. In certain embodiments, the particles are stable for at least three months at 40° C. In certain embodiments, the particles are stable for at least 12 months at 40° C. In some embodiments, the particles are stable for at least one month at 60° C.

Proteins, e.g., antibodies, are complex molecules and can have a large number of possible conformations. The three-dimensional folded state of a protein may have several thermodynamically favorable conformations, where the native state may or may not involve the energetically most favored conformation, e.g., physical degradation. Physical degradation of proteins includes conformation changes, undesirable adsorption to surfaces, denaturation, precipitation and aggregation. Factors that are essential to producing a stable protein particle formulation can include, protein concentration, pH range and buffer type, excipient type and concentration, and storage temperature in the solid state or in suspension. Ultimately, the protein particle formulation is dictated by the stability of the therapeutic dose or dosage form.

As disclosed herein, chemical degradation of proteins refers to modifications involving covalent bonds, e.g., deamidation, oxidation, hydrolysis and disulfide bond shuffling. The pH of a protein particle formulation is an important factor that can influence the rate of oxidation by changing the oxidation potential of oxidants, ionizable amino acids, and the stability of oxidation intermediates. The samples as disclosed herein, are typically analyzed for various attributes including physical stability, chemical stability, potency, various solution properties, e.g., viscosity, opalescence, reversible self-association, and the like, and the presence of visible or subvisible particles.

In certain embodiments, the methods as disclosed herein, relate to a quantitative, high throughput method for screening formulations for stability of a protein in the particles. In some embodiments, the method selects a formulation based on the stability of the protein. In certain embodiments, the method allows the selection of a formulation based on the stability of the protein.

As described herein, characterization of the stability of the protein was accomplished using size-exclusion chromatography (SEC), differential scanning fluorimetry (DSF), circular dichroism (CD), Cation Exchange Chromatography (CIEX) and Subvisible particle (SvP) analysis. In addition, preservation of activity was analyzed using flow cytometry and Antibody Dependent Cellular Cytotoxicity (ADCC) assays.

Also described herein, SEC data confirmed that no aggregate formation was observed upon screening as compared with the label formulation (aqueous mAb as an FDA approved formulation) containing 1.9% aggregates compared to the particle formulation which contained 1.8% aggregates. After 30 days of storage at 40-60° C., DSF showed less than 1° C. thermal shift across the samples. Alternatively, CD could be used to detect differences in secondary structure (beta-sheet percentage). CIEX was used to analyze charged variants of proteins as mandated by regulation (ICH Q6B), to ensure that no chemical modifications occurred during the methods described herein, and upon storage. In certain embodiments, the methods may be less prone to chemical modification upon storage than the FDA labeled formulation. This is due to the protein being more stable in the solid state as particles.

Bioactivity preservation has been demonstrated through flow cytometry assays after storage for 30 days at 40-60° C. No discernable difference was evident between FDA label formulations and those particle formulations as described herein. ADCC assays were also performed to assess the binding and effector functions of each Ab for its target through exposure of Ab incubated target cell lines.

In certain embodiments where the number of particle formulations is large, a design of experiment (DoE) approach can considerably save limited resources. In some embodiments, the data obtained from methods designed by the DoE approach produces a model of particle formulation parameters, e.g., physical or chemical stability, that cover formulation space not covered by the experimental conditions. Using the tools of statistical analysis, one skilled in the art can estimate the significance of the particle formulation factors and estimate confidence levels for an accurate predictive model allowing higher efficiency for particle formulation development. In some embodiments, the models show a dependence of protein thermostability on pH values, buffers, and protein concentrations in the presence of ions and excipients. In certain embodiments of the disclosure, the optimal combination of all particle formulation factors can be accessed for a selected protein, and any modifications in values was made according to the model. In some embodiments of the disclosure, the objectives for the methods described herein, may include one or more of the following: What should be the pH? Which buffer should be used? Which excipients should be used? Does the choice of surfactant influence aggregation rate? Does addition of anti-oxidants affect degradation rate? Are there novel excipients that have an outsized effect on protein stability? Does addition of salt affect protein stability at lower salt concentration? What is the effect of cations and/or anions? Are there combinations of pH and buffering species that can have a beneficial effect? Are there combinations of buffering species and excipients that have a stabilizing effect? Are there combinations of excipients that are more stabilizing?

The combination of pH, types of buffers, presence of ions, excipients, surfactants and the like, creates a formulation space with multiple coordinates. To cover this space completely, many samples are required, and a multiwell plate platform is well suited for this purpose. The typical multiwell platform is 96-well based, as used herein, and requires only a small volume of protein solution. In addition to using manually controlled transfer, mixing, dispensing, and removal techniques of the materials, the methods for screening of particle formulations for protein stability as described herein, can incorporate automation or robotically controlled techniques for particle formulation screening and characterization analysis. Advantageously the methods of the present disclosure can be carried out using automated and robotic material transfer systems and automated characterization for particle formulation analysis. In some embodiments, the methods for screening of particle formulations for protein stability is performed using robotics, e.g., high-throughput robotics. High-throughput robotics are particularly useful when transferring chemical reagents or agents from chemical compound libraries, stock solutions, and the such. In some embodiments, the methods for screening of particle formulations for protein stability is performed using automation. In certain embodiments, the method for screening formulations comprising particles for stability of a protein in the particles, e.g., therapeutic biologic in the particles, is automated. In some embodiments, the automated methods as disclosed herein, allows the incorporation of immediate feedback, e.g., responses to the automated and robotic system enabling algorithms to intelligently manipulate and optimize variables based on data models for optimal screening of particle formulations for protein stability.

In certain embodiments of the disclosure, the multiplexed methods as described herein, enables characterization of multiple formulations without cross-contamination. Disposable/single use process equipment eliminated the need for cleaning, resulting in significant improvements in workflow speed. The disposable process equipment also enabled manufacture of endotoxin and extrinsic particles-free microspheres that enables early pre-clinical experimentation. Unique combinations of the parameters were explored to obtain smooth, dense and spherical particles in the syringe-able range of less than 100 μm (container and process liquid volume, feed solute concentration, vortexing orbit, vortexing speed, vortexing time). In some embodiments, the front-loaded design methods for 80 formulations comprising proteins as described herein, provided certain formulation components as listed below: 5 pH levels: 5.0, 5.5, 6.0, 6.5, 7.0; 3 buffering species: Histidine·HCl, sodium succinate, sodium phosphate; 6 major excipients+1 no excipient condition: No excipient, Trehalose, Sucrose, Mannitol, Sorbitol, Arginine·HCl, Proline; 15 two-way combinations of the 6 major excipients; 3 surfactant choices: PS80, PS20, Poloxamer 188; 2 Anti-oxidants: Methionine and Pentetic acid; 7 Novel excipients: Tryptophan, hydroxypropyl beta-cyclodextrin, Isoleucine, Glycine, Threonine, Glutamine, Asparagine; 4 salts: NaCl high, NaCl low, KNO₃, MgCl₂; and Time points: TO, Week 1, Week 2, Week 4, Month 3. In certain embodiments, the front-loaded design methods for 80 formulations comprising proteins as described herein, provided 6 validated particle formulations.

Certain embodiments of this disclosure are described herein. Of course, variations, changes, modifications and substitution of equivalents of those preferred embodiments will become apparent to those of ordinary skill in the art upon reading the foregoing description. The inventors expect skilled artisans to employ such variations, changes, modifications and substitution of equivalents as appropriate, and the inventors intend for the disclosure to be practiced otherwise than specifically described herein. Those skilled in the art will readily recognize a variety of non-critical parameters that could be changed, altered or modified to yield essentially similar results. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

While each of the elements of the present disclosure is described herein as containing multiple embodiments. It should be understood that unless indicated otherwise, each of the embodiments of a given element of the present disclosure is capable of being used with each of the embodiments of the other elements of the present disclosure and each such use is intended to form a distinct embodiment of the present disclosure.

It will be understood by one of ordinary skill in the relevant arts that other suitable modifications and adaptations to the methods described herein are readily apparent from the description of the disclosure contained herein in view of information known to the ordinarily skilled artisan, and may be made without departing from the scope of the disclosure or any embodiment thereof.

### Kits

In various embodiments, a kit is envisioned comprising methods for screening particle formulations comprising at least one protein for stability. For example, a kit may include one or more additional containers and/or wells, each with one or more of various materials (such as reagents, optionally in concentrated form) desirable from a commercial and user standpoint for use of methods described herein. Non-limiting examples of such materials include, but not limited to, reagents, buffers, excipients, diluents, filters, needles, syringes, cartridges, carrier, package, container, wells, vial and/or tube labels listing contents and/or instructions for use, and package inserts with instructions for use. In some embodiments, the present disclosure contemplates a kit for screening of particle formulations for protein stability of the embodiments. The kit may comprise one or more sealed reagents, buffers, excipients, diluents, filters, needles, syringes, cartridges, carrier, package, container, wells, vial and/or tubes containing any of the materials of the present disclosure. The kit may include, for example, at least one antibody as well as reagents to prepare and formulate the components of the embodiments or perform one or more steps of the disclosed screening methods. In some embodiments, the kit may also comprise a suitable container or wells, that will not react with components of the kit, such as an Eppendorf tube, a bottle, a tube, or the like. The container or wells may be made from sterilizable materials such as plastic, glass, or the like.

The kit may further include an instruction sheet that outlines the procedural steps of the methods set forth herein, and will follow substantially the same procedures as described herein or are known to those of ordinary skill in the art. The instruction information may be a computer readable media containing machine-readable instructions that, when executed using a computer, causes the display of a real or virtual procedure for screening of particle formulations for protein stability.

A label is optionally on or associated with the container or wells. For example, a label is on a container when letters, numbers, or other characters forming the label are attached, molded or etched into the container itself. A label is associated with a container when it is present within a receptacle or carrier that also holds the container, e.g., as a package insert. In addition, a label may be used to indicate that the contents are to be used for a specific screening application. In addition, the label may indicate directions for use of the contents, such as in the methods described herein. In certain embodiments, the materials are presented in a pack or dispenser device which contains one or more-unit forms containing a protein provided herein. The pack for example contains metal or plastic foil, such as a blister pack. Or, the pack or dispenser device may be accompanied by instructions for use. Or, the pack or dispenser may be accompanied with a notice associated with the container in form prescribed by a governmental agency regulating the manufacture, use, or sale, which notice is reflective of approval by the agency.

The disclosure generically described, will be more readily understood by reference to the following examples, which are included merely for purposes of illustration of certain aspects and embodiments of the present disclosure, and are not intended to be limiting.

### EXEMPLIFICATION

### Abbreviations

Å angstrom
aa amino acids
Agg Aggregates
BSA bovine serum albumin
° C. degrees Celsius
cm centimeter
d day
DI deionized
Dim Dimer
DP dispersed phase
EA ethylacetate
ELISA enzyme-linked immunosorbent assay
EO ethyloleate
eq. equivalent
Et ethyl
eV electron-volts
FDS filtered drug substance
g gram
GC gas chromatography
h hour
HDPE high-density polyethylene
hIgG human IgG
HPLC high performance liquid chromatography
hr hour
HSA human serum albumin
ID internal diameter
IV intravenous
K thousand
KF Karl Fischer
kJ kilojoules
kPa kiloPascal
kV kilovolts
L liter
LC-MS liquid chromatograph mass spectrometry
LD laser diffraction
LDS liquid drug substance
Lyo lyophilization
m meta
mAb monoclonal antibody
MALDI-MS matrix-assisted laser desorption ionization mass spectrometry
Me methyl
min minute
μg microgram
μL microliter
μm micrometer
uM micromolar
mg milligram
mL milliliter
mm millimeter
mM millimolar
MOC micro-orifice collector
mol mole
Mon Monomer
mPa·s milliPascal·second
mTorr milliTorr
N newton
nBA n-butylacetate
nm nanometer
p para
PBS phosphate-buffered saline
PEG polyethylene glycol
PI pressure indicator
ppm parts per million
PS 80 polysorbate 80
PTFE polytetrafluoroethylene
rcf relative centrifugal force
RH relative humidity
rpm revolutions per minute
RT room temperature
S second
SC subcutaneous
sec second
SEM scanning electron microscopy
SLPM standard liter per minute
t tertiary
tert tertiary
UHMW ultrahigh molecular weight polyethylene
um micrometer
UTW ultra thin wall
UOM unit of measure
UV ultraviolet
VIS visible

### Materials

Human IgG (IRHUGGF-LY, >97%) and bovine IgG (IRBVGGF) were obtained from Innovative Research as a powder or as an aqueous solution. Bovine serum albumin (BSA) and human serum albumin (HSA) were purchased from Sigma-Aldrich. The monoclonal antibodies (mAb) were provided and received as an aqueous solution. A biosimilar of Roche's Rituximab was purchased from a vendor that provided the antibody in an aqueous composition as 10 mg/mL rituximab, 9 mg/mL sodium chloride, 7.35 mg/mL sodium citrate dihydrate, and 0.7 mg/mL polysorbate 80. A biosimilar of Roche's trastuzumab was purchased from Genscript Biotech Corporation that provided the antibody as an aqueous composition. Composition of custom “feed solutions” used for processing particles were produced through modifying the received formulation by desalting followed by concentrating and adding desired excipients or by direct buffer exchange. All excipients used in particle composition have been used in existing approved biologics injections. Concentration columns were procured from Millipore Sigma (Amicon® Ultra 15 mL Filters for Protein Purification and Concentration with a 10-50 kDa cut off) and used where necessary to: (i) reach the desired monoclonal antibody concentration, and (ii) exchange buffer/excipients before particle formation. Zeba desalting columns (THERMO FISHER SCIENTIFIC™ 87773) were also used to remove salt from solutions in certain instances. Typically, the ratio of residual salt to protein in the desalted solutions (wt/wt) was <1% as determined form conductivity measurements and/or elemental analysis. All excipients were purchased from Sigma-Aldrich and used as received.

### Methods

FLOWCAM™: Particle sizing was measured using FLOWCAM™; a dynamic image analysis instrument. Samples were diluted to about 1 mg/mL in isopropanol and passed through a thin channel. Images of particles were recorded and analyzed according to size and shape (count-weighted).

Image Analysis: Particle diameters and circularity were measured using ImageJ analysis on SEM images. The analysis was performed on, for example, at 500× or 1000× images. The ImageJ Particle Analysis tool was run on the image, identifying objects with a circularity of >0.8 and size >0.5 μm with each object outlines. These outlines were visually inspected for good fit. Any mis-identified particles were manually rejected and any missed particles were manually included and measured using the ImageJ diameter tool. Select microscopy images were chosen for further analysis on the basis of (i) minimal particle overlapping, (ii) good contrast between the particles and the background, and (iii) a resolution providing for particle occupancies of at least 10 pixels. This allowed for particles to be easily identified and reduced resolution-based error. A binary threshold was applied to separate the particles from background, and a watershed segmentation algorithm was applied to ensure that individual particles were measured separately. The ImageJ tool “Analyze Particles” was then applied on the binary picture with the following parameters: circularity between 0.5 and 1.0; size between 5 and infinity square microns; exclude on edges; fill holes. The outlines of the identified particles were overlaid onto the original image. Particles which were misidentified, such as clusters that were identified as a single particle or particles whose outlines do not match the particle, were then discarded. Missing particles were measured by manually tracing the particle's outline and using ImageJ's Measure tool.

Accelerated Storage Protocol: All samples were transferred to glass-bottom plates and 2R Schott vials for aging (typically 2 mL or 4 mL volume, depending on sample). The glass-bottom plates and 2R Schott vials were sealed with parafilm, placed in an oven at 4° C., 25° C., 40° C., 50°, or 60° C., and visually inspected over the aging period to ensure integrity and stability.

Particle Dissolution: Phosphate-buffered saline (PBS) was added to dry particle samples to produce a final concentration of 50 mg/mL (particle mass/mL of solution, 96-well plate). After a period of time, 10 μL aliquot was removed from the sample vial and the absorbance at 280 nm was measured and recorded. The Ab, e.g., mAb, concentration was plotted against time for all samples.

Size Exclusion Chromatography (SEC) Measurements: 20 μL Injections of samples (1 mg/mL) were run at a flow rate of 1 mL/min in SEC buffer (25 mM phosphate, 250 mM NaCl pH 6.8) for 15 minutes on an AGILENT™ ADVANCEBIO™ SEC (300 mm×2.7 μm, 300 Å column). Peak analysis was performed by auto-integrating using the following parameters: slope sensitivity=0.5, peak width=0, height reject=0, area reject=0, shoulders off, area percent reject 0, standard tangent skim mode, advanced baseline correction, 0 for front peak skim height ratio, 0 for tail peak skim height ratio, 0 for peak to valley ratio, and 0 for skim valley ratio. Alternatively, 20 μL injections of samples (1 mg/mL) were run at a flow rate of 1 mL/min in SEC buffer (25 mM phosphate, 250 mM NaCl pH 6.8) for 15 minutes on an AGILENT™ ADVANCEBIO™ SEC (300 mm×2.7 μm, 300 Å column). Peak analysis was performed by auto-integrating using the following parameters: slope sensitivity=0.5, peak width=0, height reject=0, area reject=0, shoulders off, area percent reject 0, standard tangent skim mode, advanced baseline correction, 0 for front peak skim height ratio, 0 for tail peak skim height ratio, 0 for peak to valley ratio, and 0 for skim valley ratio.

Scanning Electron Microscopy (SEM): Electron micrographs were collected for select samples with either a HITACHI™ TM3030Plus or a TM1000 tabletop microscope. The samples were immobilized on conductive tape and examined in a low-vacuum anti-charging environment, obviating the need for sample preparation.

Subvisible Particle (SvP) Analysis: Subvisible particles (SvPs) were analyzed with a FLUID IMAGING TECHNOLOGIES™ FLOWCAM™ PV-100 system. Samples for analysis were reconstituted in sterile centrifuge tubes with filtered water (MILLI-Q™) to the concentration of interest. Three sets of samples were investigated thereafter. These included (i) a sample of the diluent used for reconstitution, (ii) an aliquot of the feed solution used for the particle formation process, i.e., a sample of the first aqueous liquid, and (iii) the reconstituted material.

Dynamic Scanning calorimetry (DSC): Powdered samples were analyzed using dynamic scanning calorimetry. Masses of 5 to 10 mg of powdered samples were loaded into aluminum crucibles and sealed hermetically. Crucibles were loaded into the instrument, and the heat flow into the samples was monitored while the temperature was ramped from −80-200° C., optionally, from 20-180° C., at a constant rate of 5° C./minute.

USP <790>: According to the USP <790> standard, samples of dissolved particles were visually observed against a white and black background under lighting conditions greater than 2000 lux. Matte-finished high density polyethylene sheets were selected for the background to reduce glare. The illuminance at the viewing point was confirmed with a lux meter (Dr. Meter, LX1330B). The samples were swirled before being held up to the backgrounds and viewed for 5 sec.

### Example 1

Preparation of Protein Particles: 1. Protein solution with various amount of excipient was diluted to reach a final protein concentration of 30 mg/mL. 2. The resulting solution was then filtered through a small 0.22 μm syringe filter to remove any extrinsic particles from the aqueous “feed” solution. 3. 25 mL of dehydration solvent (e.g.: n-butyl acetate) was added to a 50 mL conical tube (Disposable 50 mL, polypropylene tubes). 4. 80-120 uL of protein feed was then added to each tube. 5. The tube was vortexed (Vortex Genie) for 30 seconds (speed=10, 3200 rpm and 4.9 mm orbit) until droplets were dehydrated (time dependent on the dehydration solvent). 6. To isolate protein microspheres from the dehydration solvent, the tubes were centrifuged at 100-500 g for 2 minutes. 7. Supernatant was removed by a quick inversion into a waste collector. 8. The remaining solids were resuspended in roughly 300 uL to 1 mL of the dehydration solvent and transferred to a 1.5 mL centrifuge tube. 9. The 1.5 mL tubes were centrifuged at 100-500 g for 2 mins and excess solvent was removed using a pipette. 10. Residual solvent was removed by vacuum drying. 11. Solids obtained were re-dissolved in 100-120 uL DI water and incubated in the 4° C. fridge for complete redissolution. 12. Samples were then well-mixed and analyzed to measure turbidity and protein concentration. 13. Once the turbidity has been measured, samples were diluted 10-fold using 900 uL of clean particle-free DI water. 14. Protein concentration was measured for the diluted sample followed by SvP analysis using FLOWCAM™ and soluble aggregate analysis using HPLC-SEC. The general workflow for the HTS method is shown in FIG. 1. Alternatively, a feed amount of 120 uL, centrifuge speed at 500 g, vacuum drying for 12 hours can be used. Protein concentrations in the range of 1 mg/mL to 50 mg/mL have been processed with various amount of excipient according the general method. Through pH, buffer, and excipient screening, various lead formulation candidates were identified with microparticle protein loading of at least 60 to 93% (w/w) with excipient content 7 to 40% (w/w). Formulations with up to 85% protein loading with excipient content of up to 15% showed improved stability compared to the starting aqueous feed standard solution.

### Example 2

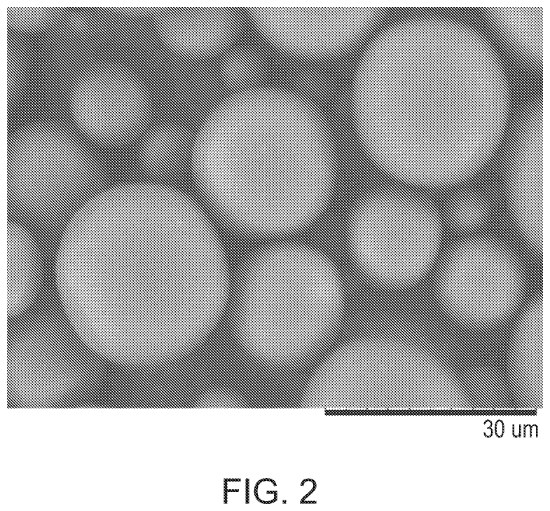

Drying: After particles were produced, aliquot the slurry of protein particles into solvent-compatible 96 well plates (Corning CLS3365). Centrifuge the plate at 500 XG for 2 minutes and remove the solvent supernatant with multichannel pipette and vacuum dry. Particle size distribution showed a mean particle size to be 26.03 μm and the median to be 25.3 μm. D10=16.30 μm, D50=25.28 μm and D90-36.65 μm were reported based on particle size analysis (FLOWCAM™), count-weighted. Particle size was also determined using a laser diffraction based particle analyzer (Horiba™ LA-960S). SEM images revealed identifiable circular particulate matter at less than about 25 μm as shown in FIG. 2. The average circularity was calculated to be approximately 0.94.

### Example 3

Redissolution: 120 uL of the redissolution media (e.g. filtered DI water) was added to the plate containing protein particle powder and then transferred to the plate reader. An absorption kinetics experiment was conducted measuring absorption at 350 nm to track the breakthrough of the protein microsphere cake over a period of 2 hours with 1 minute increments. An asymptotic leveling of the absorption signal to a relatively low level indicated complete dissolution.

USP<790>: The presence of visible particles was determined in dissolved samples of particles. USP <790> was used to determine if there were particles present in the “visible” range (>100 um). Observations of dissolved formulations were used to assess the presence of visible particles. The observations were made briefly (5 seconds) with a white and black background under the appropriate lighting. Some potential small particles were observed, but were difficult to see by eye likely classifying them as subvisible particles. Given that these particles may be considered subvisible, other methods have been used to further investigate as described below.

Sub-visible Particle (SvP) Analysis: The subvisible particle matter present in dissolved particles was investigated. Sub-visible particle analysis was performed after dissolution of the particles under standard protocol. The data were compared to a high concentration (50-200 mg/mL) liquid drug substance of the same protein (control sample). The SvP analysis was performed using a particle analyzer where the particle count was adjusted for background signal in the control sample. Under general accelerated storage, the subvisible particle counts measured in the optimized microparticle suspension samples were lower than the liquid drug substance control sample.

### Example 4

Turbidity: Prior to transferring, the sample was mixed well by pipet. 100 uL of each sample was transferred to a Costar UV-Star 96 well plate. The absorbance was measured at 350 nm and 405 nm wavelength using a Molecular Device M3 Plate Reader. The reading was repeated three times.

### Example 5

Concentration: Centrifuge the plate containing samples at 2000 XG for 3 minutes to let extrinsic particles settle. Transfer 50 uL of the protein supernatant into a 96 well UV-Star half-area plate using a multichannel pipette. Create standards including a blank on the plate. For the standard curve, at least 5 concentration points was chosen. Centrifuge the plate at max speed for 30 seconds to settle the liquid drops stuck on the wall and to create a flat liquid surface to get absorbance. Measure absorbance at 305 nm. The absorbance was measured three times. Calculate the concentration of the unknown samples using the standard curve. Dilute if necessary to ensure range linearity.

### Example 6

Chromatography: After protein concentration was determined following “Concentration” general procedure, calculate the diluent and add the calculated volume of diluent to a new filtered 96 well plate (mesh size 0.2 um). Place an HPLC compatible plate (Corning CLS3365) underneath. Use a multi-channel pipet to transfer the concentrated protein sample to the corresponding well and mix to dilute the samples to the desired concentration in SEC buffer (e.g., 1 mg/mL in PBS, 10 mg/mL in water) to a volume of at least 100 uL or 250 uL. Centrifuge at 2000 XG for 2 minutes. To minimize evaporation, a sealing mat (Life Technology) was used to seal the plate prior to placing it in the HPLC. Stored at 4° C. for 24 hours, transfer the samples to a new filtered plate and centrifuge again at 2000 XG for 2 minutes prior to HPLC. Set up the sequence table for SEC and run. Collect SEC and SEC-MALS data.

### Example 7

Storage: After the protein samples were quantified following “Concentration” general procedure, transfer from the UV-STAR plate to the Corning round bottom plate. Seal plate with sealing mat (Life Technology) or parafilm. Store the plate at 4° C. For further CIEX or SEC-MALS studies: centrifuge the plate at 2000 XG for 1 minute to remove the condensation. Dilute samples to the target concentration in appropriate buffer. Filter in a filtered 96 well plate and run experiment.

### Example 8

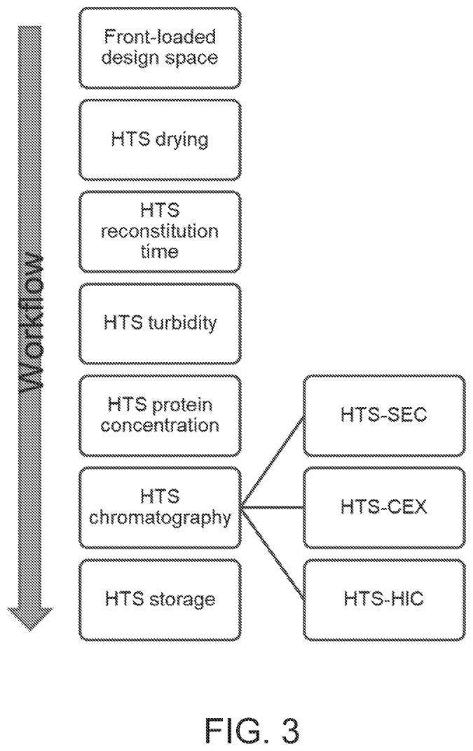

Analytical Workflow: An overview of the HTS analytical workflow is shown in FIG. 3. Example 8 was performed according to Examples 1-7. As described in the previous Examples, the general analytical workflow reduces the analysis time by employing a combination of plate-based assays and multichannel pipette transfers.

### Example 9

### Particle Quality Studies

Design: The stability of the protein particles was assessed over the course of up to three months at 5, 25, 40, 50 and 60° C. (150-300 mg/mL protein concentration) to determine the stability of the particles in comparison to the protein liquid drug substance (LDS).

Buffer Exchange by Tangential Flow Filtration (TFF): Protein feed solutions were prepared by diafiltration into the required buffer cocktails. A KROSFLOW™ KR2i TFF system (REPLIGEN™) equipped with a hollow fiber filter module (MIDIKROS™ Sampler) was used to perform the feed preparation. A number of diafiltration volume exchanges were performed with the appropriate buffer for each formulation.

SEM Imaging: The particles were imaged using a scanning electron microscope (HITACHI™, TM-1000). A sample of the particles was mounted onto an adhesive stage for analysis. Images were captured at varying magnifications using an accelerating voltage of 15 kV.

Karl Fischer Coulometry: Testing for moisture content was performed by Karl Fischer analysis using a MetroOhm (899 coulometer) equipped with an 860 KF Thermoprep oven. Particles were heated to 165° C. in an oven and the released water was determined coulometrically.

Particle Dissolution: Water was added to dry particle samples, 96-well plate, to produce a final protein concentration. The terminal dissolution concentration was recorded by removing an aliquot from the sample and measuring the absorbance at 280 nm (using the extinction coefficient E1%=1.69 L·g−1·cm−1).

Turbidity: An aliquot of reconstituted particle solution was transferred to a 1-cm path length cuvette. The absorbance at 405 nm was recorded using a NanoDrop™ One UV-VIS spectrophotometer (THERMO SCIENTIFIC™).

Size-Exclusion Chromatography (SEC) Measurements: Injections of redissolved particle samples were run at a flow rate of about 0.35 mL/min using a mobile phase comprised of 100 mM sodium phosphate monobasic and 200 mM L-arginine monohydrochloride, pH 6.5 for 10 minutes on a Tosoh TSKgel SuperSW mAb HTP (4.6 mm ID×15 cm L) column. Peaks were manually inspected to ensure accurate identification and analysis was performed by autointegration using parameters known in the art.

Strong Cation-Exchange Chromatography (SCEX): Injections of redissolved particle samples were run at a flow rate of 0.2 mL/min using a gradient method that starts at 100% mobile phase pH gradient buffer A to 100% mobile phase pH gradient buffer B followed by washing and re-equilibration for a total run time of 40 minutes on a MABPAC™ SCX-10 RS Analysis Column, 2.1 mm ID×15 cm L, 5 μm column. Peaks were manually inspected to ensure accurate identification and analysis was performed by autointegration using parameters known in the art.

Hydrophobic Interaction Chromatography (HIC): Injections of particle samples dissolved in diluent comprised of 750 mM Ammonium Sulfate, 50 mM Sodium Phosphate Monobasic Dihydrate, pH 6.0 (1 mg/mL) were run at a flow rate of 1 mL/min using a gradient method that starts at 50% mobile phase A comprised of 2M ammonium sulfate, 50 mM sodium phosphate monobasic dihydrate, pH 6.0 to 100% mobile phase B comprised of 50 mM sodium phosphate monobasic dihydrate, pH 6.0 followed by washing and re-equilibration using 50% mobile phase A for a total run time of 60 minutes on a MABPAC™ HIC-20 HPLC Column, 5 μm, 4.6 mm ID×25 cm L column. Peaks were manually inspected to ensure accurate identification and analysis was performed by autointegration using parameters known in the art.

Protein A Chromatography (ProA): Injections of particle samples diluted in water to 1 mg/mL were run at a flow rate of 0.5 mL/min using a gradient method that starts at 100% mobile phase A comprised of 10 mM tris pH 7.0, 150 mM Sodium Chloride to 100% mobile phase B comprised of 20 mM sodium citrate pH 3.0, 150 mM sodium chloride followed by washing and re-equilibration using 100% mobile phase A for a total run time of 40 minutes on a POROS™ A column, 20 μm, 4.6 mm ID×5 cm L. Peaks were manually inspected to ensure accurate identification and analysis was performed by autointegration using parameters known in the art.

Particle Sizing (Laser Diffraction): Particle size analysis was conducted via laser diffraction using a Horiba™ LA-960S. Dry particles were suspended in isopropyl alcohol at a concentration of approximately 0.1 mg/mL. The particle samples were sonicated within the particle measurement instrument to ensure homogeneity and then circulated and agitated by the Horiba™ particle size analyzer. Particle size analysis was conducted using a mobile phase of isopropyl alcohol and the volume average particle size distribution was calculated.

Microflow Imaging: Flow imaging microscopy (FLOWCAM™, FLUID IMAGING TECHNOLOGIES™) was performed to quantify subvisible particulates in protein LDS and the particle formulation. Particles were first redissolved using the particle dissolution method described above and diluted to 1 mg/mL with ultrapure water. For analysis, the aqueous sample was introduced at a flow rate of 0.15 mL/min. The resulting particle counts are recorded and reported per mg of protein.

Results: The stability of dry particles were evaluated alongside the protein liquid drug substance (LDS). Stability was tracked at 5, 25, and 40° C. with data collected at 7, 14, 28 (50-60° C.), 60 and 90 days. Particle stability and protein stability were measured at each time and temperature. Analysis of the particles confirmed that protein quality remained constant as measured by the monomer profile (SEC), maintenance of charge variant profile (SCEX), isoforms/presumed oxidation (HIC, ProA), and colloidal stability (turbidity and subvisible particles). In each case, the protein feed solution and the LDS was measured against the particles. Particles stored as dry powder at 5, 25, and 40° C. showed a smooth, spherical morphology. Over the course of three months at these temperatures, no change in the particle morphology or the particle size distribution was observed. The moisture content of the particles remained constant for all storage temperatures and length of time. Analysis of protein aggregates were measured by SEC up to 90 days at 5, 25, and 40° C. The rate of aggregation for the particles demonstrated improved stability as compared to protein LDS. The charge variant profile of protein was measured by SCEX up to 90 days at 5, 25, and 40° C. No appreciable change was observed for particles, which demonstrated improved stability at 40° C. as compared to protein LDS. HIC and ProA were used to measure potential oxidation of protein. The degree of presumed global oxidation of protein was measured by HIC and ProA up to 90 days at 5, 25, and 40° C. No appreciable change was observed indicating that the particles demonstrated improved stability with respect to oxidative stress at 25 and 40° C. as compared to protein LDS at the same temperatures and times. To further probe the protein quality of the redissolved particles, visible and subvisible particulates were analyzed after storage at various temperatures. Upon dissolution, the protein solutions were essentially free of visible particulates and subvisible particulates after storage at 40° C. up to 90 days.

### Example 10

### Determination of Aggregation, Fragmentation and Change in Charge Variants

SEC: Dry powder samples were dissolved to 5% (w/v) in ultrapure water and shaken at 60 RPM. After determining triplicate protein concentrations, the samples were diluted to 1 mg/mL with PBS and syringe filtered into HPLC vials. All samples were analyzed using an AGILENT™ 1260 INFINITY II BIO-INERT™ LC System and TSKgel SuperSW HTP column (4 μm, 4.6 mmID×150 mmL) column equilibrated with 200 mM Arginine-HCl, 100 mM Sodium Phosphate pH 6.5. The autosampler and column compartment were maintained at 4° C. and 20° C., respectively, and UV absorbance was monitored at 280 nm. The run time was 10 minutes. The % areas of the integrated high molecular weight (aggregates), monomer and low molecular weight peaks were reported.

SCEX: Dry powder samples were dissolved to 5% (w/v) in ultrapure water and shaken at 60 RPM. After determining triplicate protein concentrations, the samples were diluted with ultrapure water and syringe filtered into HPLC vials. All samples were analyzed using an AGILENT™ 1260 INFINITY II BIO-INERT™ LC System and THERMO SCIENTIFIC™ MABPAC™ SCX-10 RS column (5 μm, 2.1 mmID×150 mmL) equilibrated with 100% mobile phase A (THERMO SCIENTIFIC™ 1X CX-1 pH Gradient Buffer A). The gradient is 0-20% mobile phase B (THERMO SCIENTIFIC™ 1X CX-1 pH Gradient Buffer B). The autosampler and column compartment were maintained at 4° C. and 30° C., respectively, and UV absorbance was monitored at 280 nm. The % areas of the integrated acidic, neutral (main) and basic peaks were reported.

### Example 11

Determination of Protein Content: Suspensions were redispersed by vortexing, diluted to 2.5% (v/v) in ultrapure water and shaken at 60 RPM for 30 minutes; corresponding dry powder samples were dissolved to 5% (w/v) in ultrapure water and shaken at 60 RPM for 30 minutes. After determining triplicate protein concentrations (from the bottom or aqueous layer for suspensions), the % mass of protein in particles was calculated by dividing the product of the averaged re-dissolved protein particle concentration and redissolution volume by the mass of microparticles. The averaged concentration of suspensions (corrected by aqueous/formulation carrier liquid dilution factor) is then divided by this % mass of protein in particles to calculate the theoretical mass of particles in suspension.

### Example 12

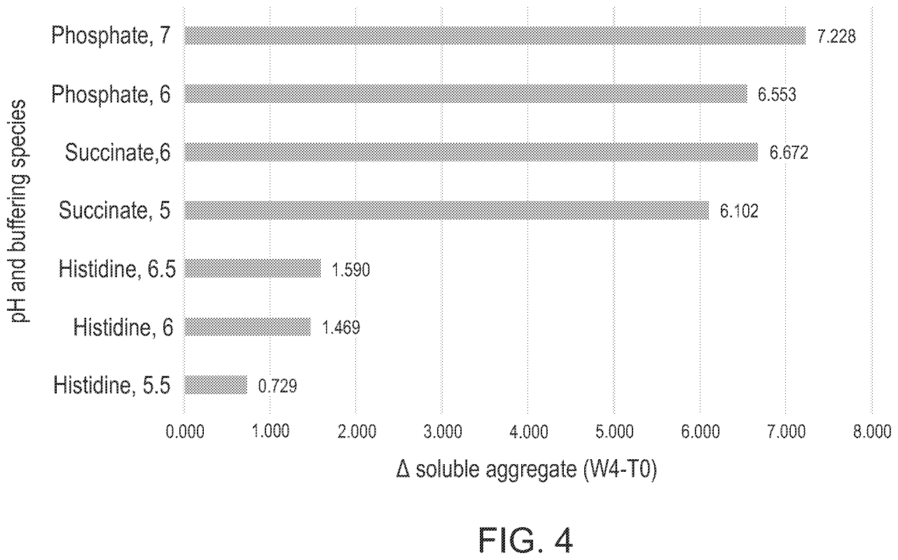

The effect of pH and buffering species selection on the stability of BSA microspheres during storage at 40° C. over 4 weeks (W4) versus TO is shown in FIG. 4. The formulations were composed of 40 mg/mL BSA, 1.55 mg/mL of indicated buffer and 4.2 mg/mL of trehalose. The composition represents an 86% (w/w) Particle Protein Percent (PPP) as weight percent. The only difference amongst the formulations is the choice of pH and buffering species. Histidine buffered formulations at pH 6 were more stable than either succinate or phosphate buffered formulations at the same pH. pH 5.5 was observed to be more stabilizing compared to pH 6.0 or 6.5.

### Example 13

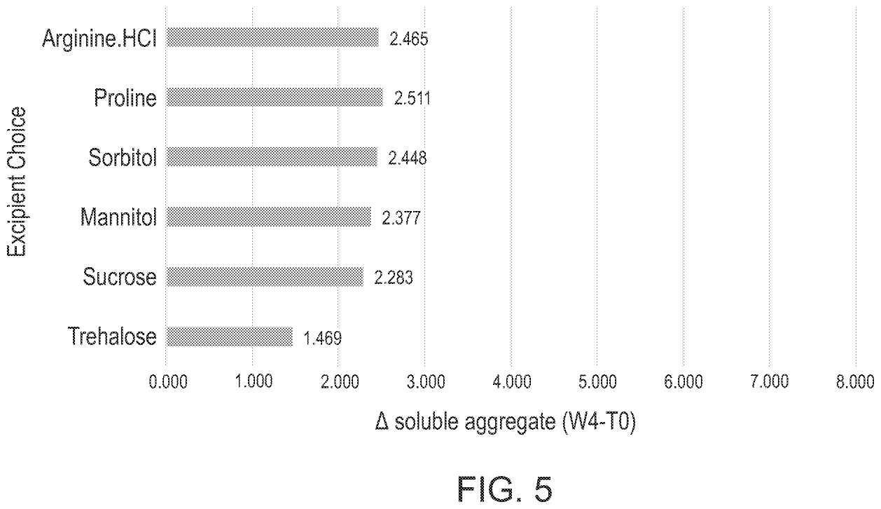

The effect of excipient selection on the stability of BSA microspheres during storage at 40° C. over 4 weeks (W4) versus TO is shown in FIG. 5. The formulations were composed of 40 mg/mL BSA, 1.55 mg/mL of Histidine buffer at pH 6.0 and 4.2 mg/ml of the indicated excipient. The composition represents an 86% (w/w) Particle Protein Percent (PPP) as weight percent. The only difference amongst the formulations is the choice of excipient. Trehalose containing formulations provided the best stability.

### Example 14

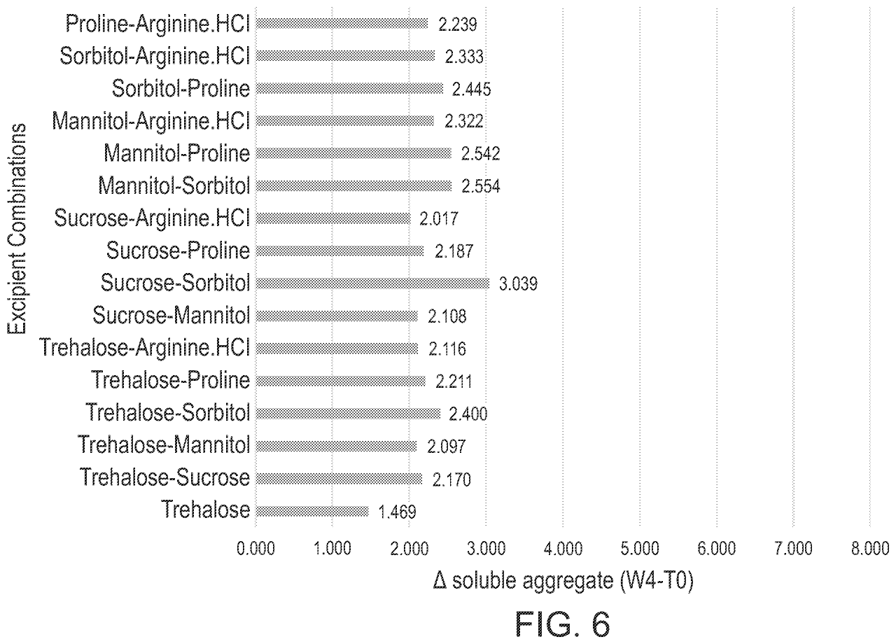

The effect of excipient combinations on the stability of BSA microspheres during storage at 40° C. over 4 weeks (W4) versus TO is shown in FIG. 6. The formulations were composed of 40 mg/mL BSA, 1.55 mg/mL of Histidine buffer at pH 6.0 and 2.1 mg/mL each of the indicated excipient. The composition represents an 86% (w/w) Particle Protein Percent (PPP) as weight percent. The only difference amongst the formulations is the combination of excipients. Trehalose containing formulations provided the best stability over all other combinations.

### Example 15

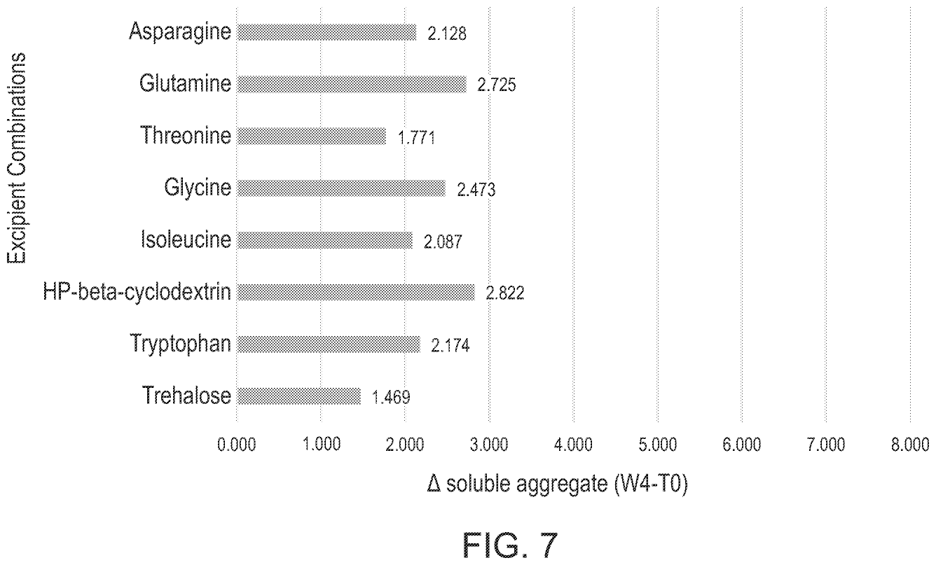

The effect of excipient selection on the stability of BSA microspheres during storage at 40° C. over 4 weeks (W4) versus TO is shown in FIG. 7. The formulations were composed of 40 mg/mL BSA, 1.55 mg/mL of Histidine buffer at pH 6.0 and 4.2 mg/ml of the indicated excipient. The composition represents an 86% (w/w) PPP as weight percent. The only difference amongst the formulations is the choice of excipient. Trehalose containing formulations provided the best stability.

### Example 16

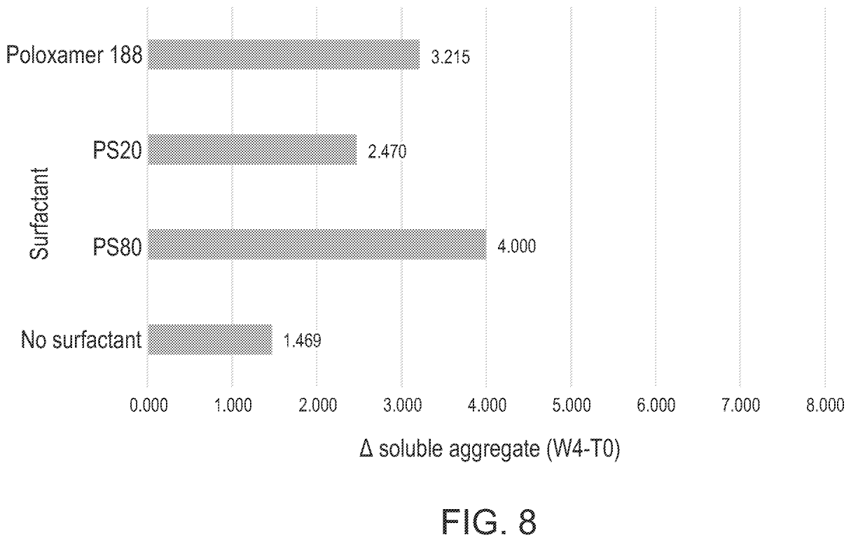

The effect of excipient selection on the stability of BSA microspheres during storage at 40° C. over 4 weeks (W4) versus TO is shown in FIG. 8. The formulations were composed of 40 mg/mL BSA, 1.55 mg/mL of Histidine buffer at pH 6.0 and 4.2 mg/mL of trehalose and 0.5 mg/mL of the indicated surfactant. The composition represents an 86% (w/w) PPP as weight percent. The only difference amongst the formulations is the choice of surfactant. Trehalose containing formulations with no surfactant provided the best stability against soluble aggregate formation. Among the surfactants, PS20 resulted in the least amount of soluble aggregate formation over 4-week storage at 40° C.

### Example 17

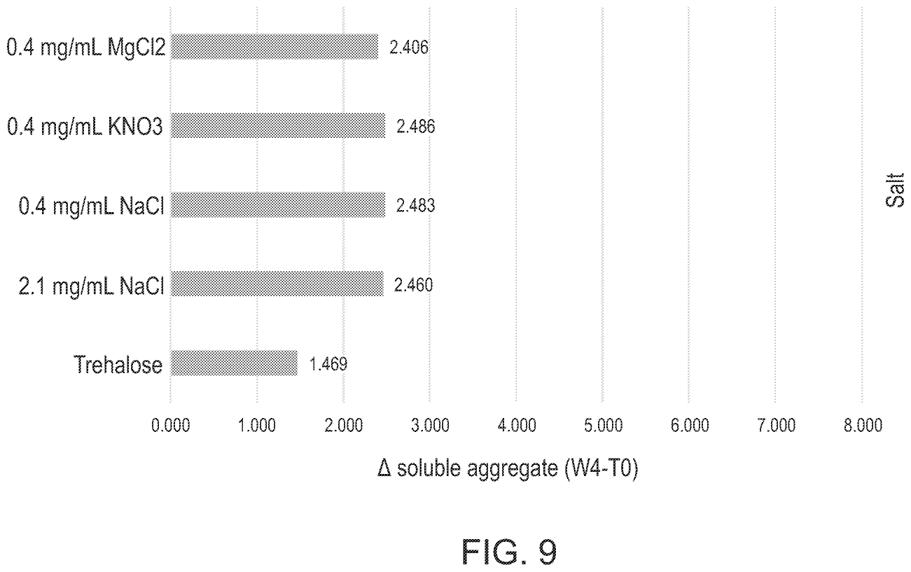

The effect of excipient selection on the stability of BSA microspheres during storage at 40° C. over 4 weeks (W4) versus TO is shown in FIG. 9. The formulations were composed of 40 mg/mL BSA, 1.55 mg/mL of Histidine buffer at pH 6.0 and 4.2 mg/mL of an excipient mixture of trehalose (3.8 and 2.1) and the indicated amount of salt (0.4 and 2.1). The composition represents an 86% (w/w) PPP as weight percent. The only difference amongst the formulations is the presence of salts. Trehalose containing formulations with no salt addition provided the best stability against soluble aggregate formation.

### Example 18

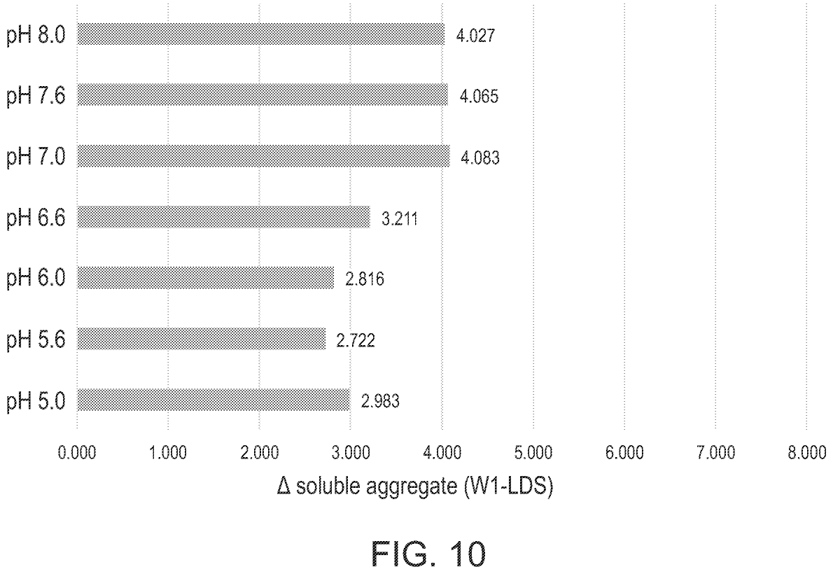

Example 18 was generally performed according to Example 12. The effect of pH selection on the stability of Rituximab microspheres during storage at 40° C. over 1 week is shown in FIG. 10. The formulations were composed of 30 mg/mL Rituximab and 10 mM of the citrate-phosphate buffer at the indicated pH. Formulations at pH 5.0, 5.6 and 6.0 were found to be the most stable.

### Example 19

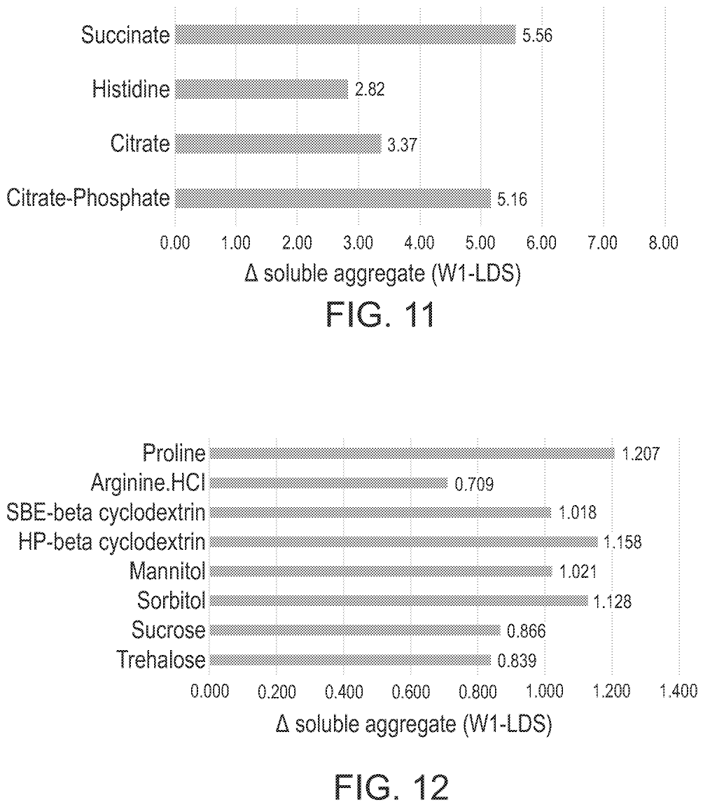

Example 19 was generally performed according to Example 12. The effect of buffering species selection on the stability of Rituximab microspheres during storage at 40° C. over 1 week is shown in FIG. 11. The formulations were composed of 30 mg/mL Rituximab and 10 mM of the indicated buffer at pH 5.6. Formulations with Histidine buffer were found to be the most stabilizing.

### Example 20

Example 20 was generally performed according to Example 13. The effect of excipient selection on the stability of Rituximab microspheres during storage at 40° C. over 1 week is shown in FIG. 12. The formulations were composed of 30 mg/mL Rituximab and 10 mM of Histidine buffer at pH 5.6 and 8 mg/mL of the indicated excipient. Formulations with Histidine buffer were found to be the most stable.

### Example 21

hIgG Screening: The following components were chosen for further optimization using DoE and model data: 10 mM Histidine buffer at pH 6.0; Proline; Trehalose; HP-beta-cyclodextrin; and NaCl at various amounts in the range of 0 to 24 mg/mL and hIgG concentration in the range of 36 to 60 mg/mL, such that the total non-volatile feed solute concentration was 61.6 mg/mL in each case. The selected protein fractions (actual protein content assuming 2% residuals and factoring in buffer contributions) were: 0.92 (87.9%); 0.98 (93.6%). A delta (dimer+agg) % at day 0 and day 7 was used as the optimization response variable. Proline was identified as the top excipient. A mixture of Trehalose and HP-betadex was identified as the second-best combination. A two analysis protocol (Mon+Dimer and Agg+Dimer %) was used to identify alternative pH and excipient choices.

### Example 22

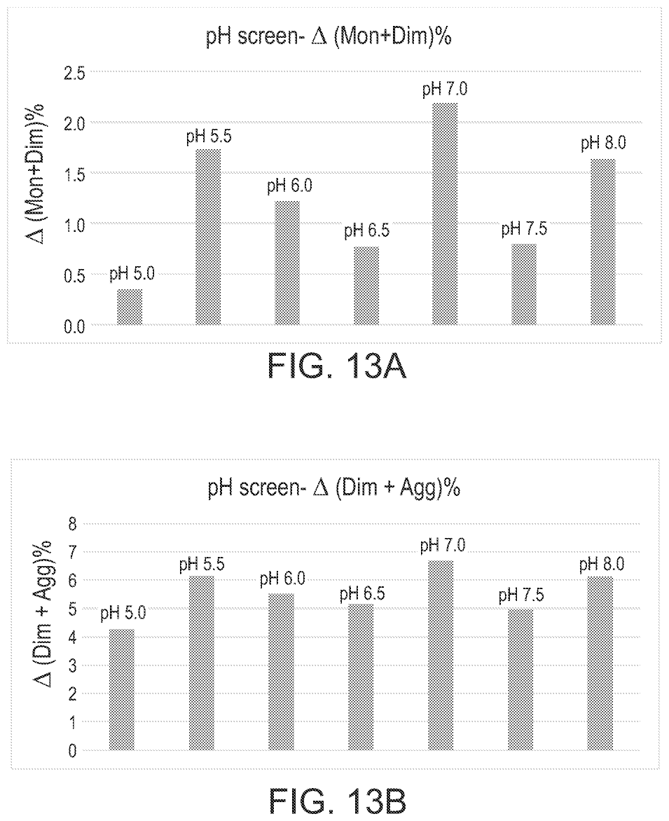

Example 22 was generally performed according to Example 21. The effect of pH selection on the stability of hIgG microspheres versus Δ (Mon+Dim) % (4 (Monomer+Dimer) %) and A (Dim+Agg) % (A (Dimer+Aggregates) %) is shown in FIGS. 13A-13B. pH 5.0, pH 6.5 and pH 7.5 showed better stability than at pH 6.0.

### Example 23

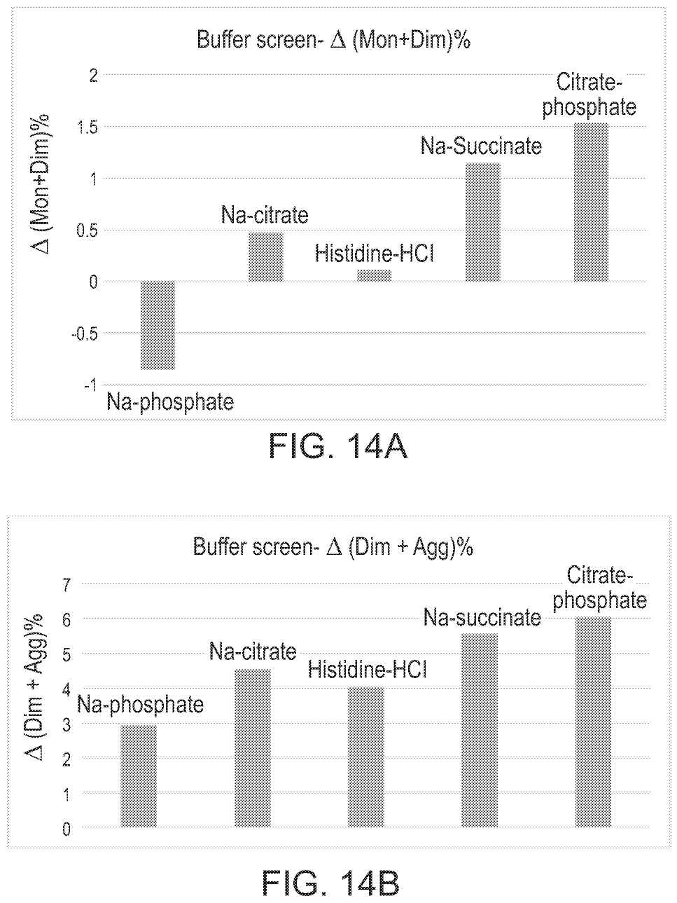

Example 23 was generally performed according to Example 21. The effect of buffer selection on the stability of hIgG microspheres versus Δ (Mon+Dim) % and Δ (Dim+Agg) % is shown in FIGS. 14A-14B. Sodium phosphate showed better stability than the Histidine buffer.

### Example 24

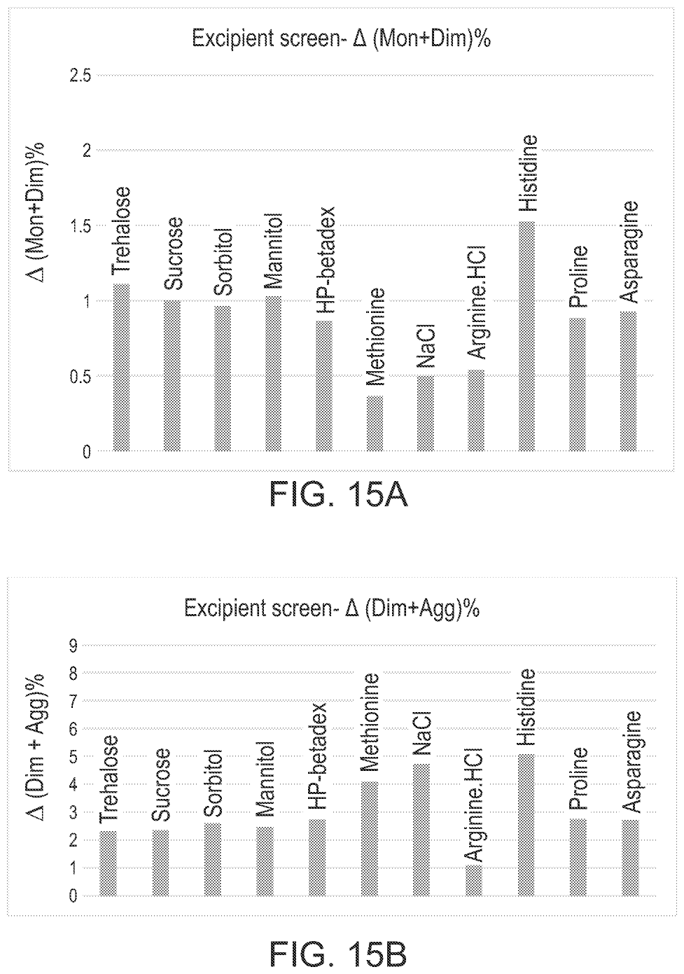

Example 24 was generally performed according to Example 21. The effect of excipient selection on the stability of hIgG microspheres versus Δ (Mon+Dim) % and Δ (Dim+Agg) % is shown in FIGS. 15A-15B. Arginine·HCl generally showed improved stability compared to Methionine, NaCl and Proline.

### Example 25

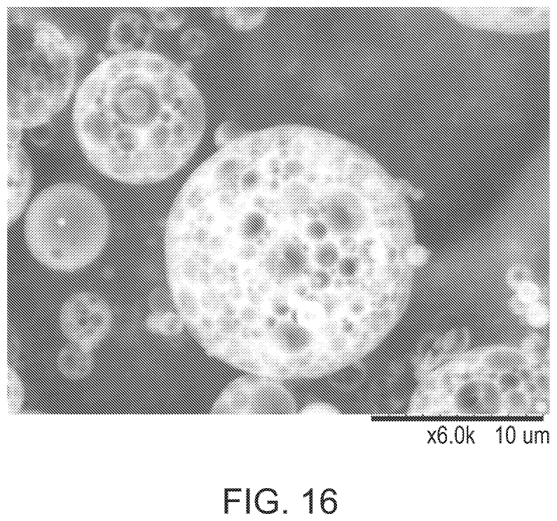

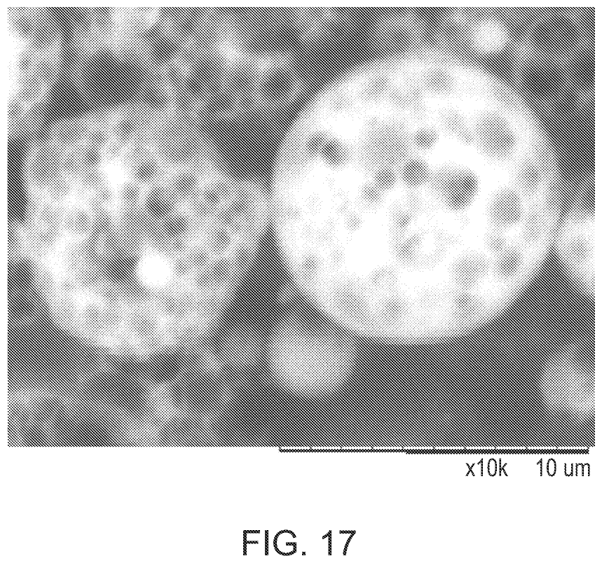

Ultrasonic Mixing: Ultrasonic mixing was used to generate particles according to Example 1. Feed solution: 40 mg/mL protein; 23.45 mg/mL L-Arginine HCl. Total feed solute loading 65 mg/mL; Particle Protein Percent (PPP) as weight percent=61.5%. Processing: DP:CP=1:200; 1 μL feed in 200 μL n-BA and 5 μL feed in 1 mL n-BA, in 2 mL low protein binding centrifuge tube. Particles produced using ultrasonic mixing at 1 μL of feed in 200 μL of n-BA is shown in FIG. 16. SEM micrographs showed circular particles at less than about 15 μm (FIG. 16). The average circularity was calculated to be approximately 0.95. Particles produced using ultrasonic mixing at 5 μL of feed in 1 mL of n-BA is shown in FIG. 17. SEM micrographs showed circular particles at less than about 10 μm (FIG. 17). The average circularity was calculated to be approximately 0.89.

### Example 26

The Effect of pH in Formulation and Aggregation of Rituximab. The protocol is to identify the optimal pH within the 5 to 8 pH range that minimizes protein aggregation during dehydration for Rituximab both at Day 0 and Day 7 (accelerated stability condition at 40-60° C.). Methodology: Rituximab stock at 16.7 mg/mL was de-salted using zeba columns followed by concentration of Rituximab in DI water to 33.3 mg/mL using Amicon columns. Next, stock solutions of 200 mM sodium phosphate, dibasic and 100 mM of citric acid were prepared. Different pH values for the citrate phosphate buffer at 100 mM strength were obtained by mixing different volumes of the stock buffers and DI water to obtain stock buffers. These buffers were then characterized with a pH meter to obtain the experimental pH values at room temperature. 630 uL of stock Rituximab in DI water at 33.33 mg/mL was then mixed with 70 uL of buffer to obtain 700 uL of final Rituximab solution at the desired pH in 10 mM citrate-phosphate buffer. The resulting solution was filtered with a 0.22 um syringe filter to obtain a particle free “feed”. 80 uL of each Rituximab feed was then dehydrated using the mixing method. 6 tubes were prepared for each pH condition. After dehydration, 3 tubes out of the 6 tubes were pooled together to constitute the day 1 sample and the remaining 3 tubes were pooled together to form the day 7 sample. The dehydrated protein samples were then vacuum dried for 16 hours on a bench-top Frogvac unit. The samples for day 7 were wrapped with parafilm and stored in an aluminum pouch to prevent ingress of moisture and stored in the 40-60° C. oven for a period of 7 days post-drying. The tests were then conducted on the dried samples both at Day 0 and Day 7: Morphology accessed using Scanning Electron Microscopy: Dried particles were observed at 100× and 300× zoom. Turbidity using Nanodrop Cuvette method: Samples were dissolved using 100 uL of water for 2 hours at 4° C. A280 readings were measured followed by Turbidity readings were recorded at 350 nm and 405 nm. Turbidity values were normalized to 30-50 mg/mL. Soluble aggregates using HPLC-SEC: Samples post turbidity measurements were collected back from the cuvette and diluted to 1 mg/mL. The TOSOH mAb HTP method was used to run SEC. Subvisible particles using FLOWCAM™: Samples post turbidity measurements were collected back from the cuvette and diluted to 2 mg/mL. Turbidity values were recorded on day 1. Conclusions: Based on the data from day 1 and day 7 it was found that pH 5.6 was the best result both on immediate analysis and 7-day stability analysis as per the percentage of soluble aggregates. This pH will be used to investigate the effect of buffering species in Example 27.

### Example 27

The Effect of Buffering Species in Formulation and Aggregation of Rituximab: The protocol is to identify the choice of a buffering species that affects protein aggregation during the dehydration step for Rituximab. Methodology: Rituximab stock at 16.7 mg/mL was concentrated using Amicons and then desalted using zeba columns to obtain a final concentration of 33.33 mg/mL. Stock buffers (histidine/histidine·HCl, citric acid/sodium citrate, succinic acid/sodium succinate, and citrate-phosphate) were prepared at 100 mM strength. 630 uL of stock Rituximab in DI water at 33.33 mg/mL was then mixed with 70 uL of buffer to obtain 700 uL of final Rituximab solution at the desired pH of 5.6 for the buffering species. The resulting solution was filtered with a 0.22 um syringe filter to obtain a particle free “feed”. 80 uL of each Rituximab feed was then dehydrated using the mixing method. 6 tubes were prepared for each buffering species. After dehydration, 3 tubes out of the 6 tubes were pooled together to constitute the day 1 sample and the remaining 3 tubes were pooled together to form the day 7 sample. The dehydrated protein samples were then vacuum dried for 16 hours on a bench-top Frogvac unit. The samples for day 7 were wrapped with parafilm and stored in an aluminum pouch to prevent ingress of moisture and stored in the 40-60° C. oven for a period of 7 days post-drying. Tests were then conducted on the dried samples both at Day 0 and Day 7: Morphology accessed using Scanning Electron Microscopy: Dried particles were observed at 100× and 300× zoom. Turbidity using Nanodrop Cuvette method: Samples were dissolved using 100 uL of water for 2 hours at 4° C. A280 readings were measured followed by Turbidity readings were recorded at 350 nm and 405 nm. Turbidity values were normalized to 30-50 mg/mL. Soluble aggregates using HPLC-SEC: Samples post turbidity measurements were collected back from the cuvette and diluted to 1 mg/mL. The TOSOH mAb HTP method was used to run SEC. Based on day 7 data for soluble aggregates and turbidity change Histidine buffer at pH 5.74 was found to be the most stabilizing buffering species.

### Example 28

The Effect of Excipients in Formulation and Aggregation of Rituximab: The protocol was to identify stabilizing excipients at the chosen pH and buffering condition that affects protein aggregation for Rituximab. Methodology: Rituximab stock at 15 mg/mL was de-salted using zeba columns followed by concentration of Rituximab in DI water to 30 mg/mL using Amicon columns. Stock buffers (histidine/histidine·HCl) for pH 5.0-6.5 were prepared at 100 mM strength. Stock solutions were also prepared for the excipients tested in this study. The sugars trehalose, sucrose, sorbitol, mannitol, HP-beta-cyclodextrin and Sulfo Butyl ether Beta-cyclodextrin were prepared at a stock concentration of 80 mg/mL and diluted 10-fold to obtain a final formulation loading of 8 mg/mL. The amino acids L-arginine·HCl, proline and lysine were prepared at a stock concentration of 80 mg/mL and diluted 10-fold to a final concentration of 8 mg/mL. The amino acid asparagine was prepared at 20 mg/mL and diluted 2.5-fold to reach a final concentration of 8 mg/mL. Rituximab samples containing the stock buffer and indicated excipient were prepared to obtain roughly 500 uL of Rituximab “feed”. The feed solution was filtered with a 0.22 um syringe filter to obtain clean “feed” for subsequent dehydration. 4 Tubes containing 80 uL each of the Rituximab feed were dehydrated using the PIT method for each condition. The 4 tubes were pooled together and divided into two 1.5 mL centrifuge tubes for analysis on Day 0 and Day 5. The dried powder was re-dissolved in 80 uL of fresh and particle free DI water. Tests were then conducted on the dried samples both at Day 0 and Day 7: Morphology accessed using Scanning Electron Microscopy. Soluble accessed aggregates using HPLC-SEC. Subvisible particles using FLOWCAM™. Turbidity accessed using Nanodrop Cuvette method. Based on the data from Day 1 and Day 7, Sucrose and Arginine. HCl was found to be the most stabilizing excipients among the excipients tested.

### Example 29

The Effect of Excipients in Formulation and Aggregation of Trastuzumab: Example 29 was generally performed according to Example 28. Formulation components: Buffer (Histidine·HCl, pH 6.0); Excipients (Trehalose, Sucrose, Sorbitol, Arginine·HCl, Glycine at 26 mg/mL); Surfactants (PS20 at 0.01, 0.1, 0.5 mg/mL and PS80 at 0.5 mg/mL); Feed solute content (60 mg/mL, 100 mg/mL); for a Protein content of about 70%. Particles were stored at 40-60° C., sealed in aluminum pouches, and the feed was stored at 4° C. (D0-2W). For a target protein content of 70%: Solute loading of 100 mg/mL showed optimized turbidity for both processing and storage; 2.58 mg/mL of Histidine at pH 6.0 was found to be the optimal buffering species; 26 mg/mL of Arginine. HCl was found to be the most stabilizing excipient based on turbidity and SEC studies for both processing and storage (2W storage at 40-60° C. with about 2.1% of solute aggregates); PS80 at 0.1 mg/mL was found to be the most stabilizing surfactant based on turbidity and SEC studies for both processing and storage; and a feed solution of 71.4 mg/mL protein concentration was found to be the optimal concentration for both processing and storage.

### Example 30

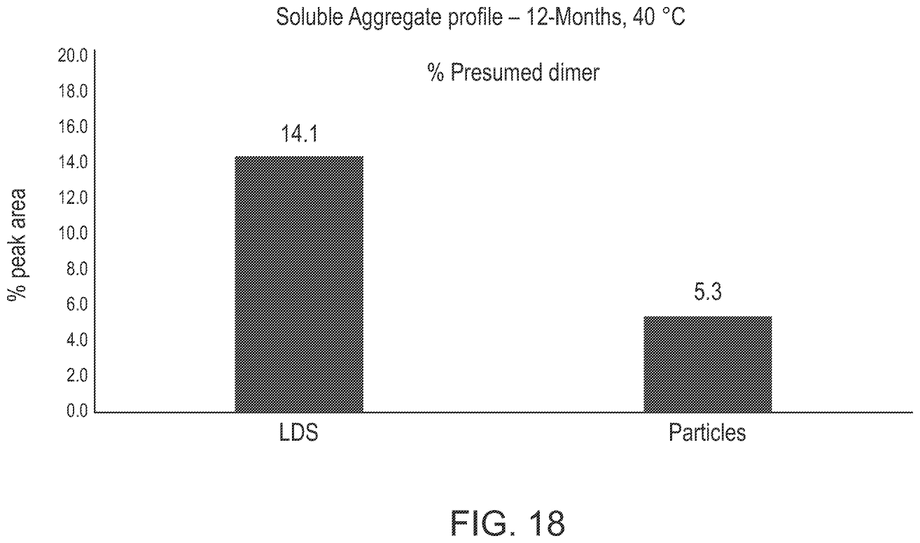

Example 30 was generally performed according to Example 9 and Example 21. The effect of excipient selection (histidine (10 mM, pH 5.5), arginine·HCl (19.5 mg/mL), and PS80 (0.05 w/v %)) on the stability of the mAb (IgG) (45.5 mg/mL) microspheres versus Δ (Mon+Dim) % and Δ (Dim+Agg) % was conducted and stored at 40° C. for 12 months to assess aggregation over time when compared to a LDS sample. As shown in FIG. 18, LDS sample stored at 40° C. for 12 months demonstrated high protein aggregation in comparison to the microsphere sample stored at the same temperature (40° C. for 12 months).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific aspects and embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A method for screening formulations comprising particles for stability of a protein in the particles, comprising the steps of:
a) dispensing a plurality of different mixtures into a plurality of wells, each mixture comprising a quantity of particles, a first aqueous liquid, and an organic liquid, wherein the particles comprise a protein and one or more pH adjusting agents, and the circularity of the particles is from about 0.80 to about 1.00;
b) removing the first aqueous liquid and the organic liquid from each well to provide a formulation comprising particles in each well;
c) dissolving the particles in a second aqueous liquid and determining the stability of the protein in each well after the first aqueous liquid and the organic liquid have been removed; and
d) selecting a formulation based on the stability of the protein determined in c).

2. The method of claim 1, wherein the mixture comprising a quantity of particles, a first aqueous liquid and an organic liquid in a) is prepared by:
i. adding a quantity of a protein in a first aqueous liquid into containers to provide a plurality of containers, each container comprising a quantity of the protein in the first aqueous liquid;
ii. adding one or more pH adjusting agents to each container;
iii. adding an organic liquid to each container; and
iv. mixing the first aqueous liquid comprising the protein and the pH adjusting agent with the organic liquid in each container, thereby preparing the mixture comprising a quantity of particles, a first aqueous liquid, and an organic liquid in each container, wherein the particles comprise the protein and one or more pH adjusting agents.

3. The method of claim 1, wherein the protein is a therapeutic biologic.

4. The method of claim 1, wherein the protein is BSA or HSA.

5. The method of claim 3, wherein the therapeutic biologic is an antibody or fragment thereof.

6. The method of claim 5, wherein the antibody is a human antibody.

7. The method of claim 6, wherein the human antibody is an IgG antibody.

8. The method of any one of claims 5-7, wherein the antibody is a monoclonal antibody.

9. The method of claim 3, wherein the therapeutic biologic is a fragment of an antibody.

10. The method of any one of the preceding claims, wherein the first aqueous liquid is water, 0.9% saline, lactated Ringer's solution, or dextrose 5%.

11. The method of any one of the preceding claims, wherein the first aqueous liquid is water.

12. The method of any one of the preceding claims, wherein the second aqueous liquid is water, 0.9% saline, lactated Ringer's solution, or dextrose 5%.

13. The method of any one of the preceding claims, wherein the second aqueous liquid is water.

14. The method of any one of the preceding claims, wherein the containers comprises tubes, vials, ampules, envelopes, flasks, or bottles.

15. The method of any one of the preceding claims, wherein the pH adjusting agent is acetate, citrate, glutamate, glycinate, histidine, lactate, maleate, phosphate, succinate, tartrate, bicarbonate, aluminum hydroxide, phosphoric acid, hydrochloric acid, sulfuric acid, DL-lactic/glycolic acids, phosphorylethanolamine, tromethamine, imidazole, glyclyglycine, monosodium glutamate, sodium hydroxide, potassium hydroxide, sodium phosphate, or a combination thereof.

16. The method of any one of the preceding claims, wherein the organic liquid is acetonitrile, chlorobenzene, chloroform, cyclohexane, cumene, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, methylisobutylketone, N-methylpyrrolidone, nitromethane, pyridine, sulfolane, tetrahydrofuran, tetralin, toluene, 1,1,2-trichloroethene, xylene, acetic acid, acetone, anisole, 1-butanol, 2-butanol, butylacetate, tert-butylmethyl ether, dimethyl sulfoxide, ethanol, ethylacetate, ethyl ether, ethyl formate, formic acid, heptane, isobutylacetate, isopropylacetate, methylacetate, 3-methyl-1-butanol, methylethyl ketone, 2-methyl-1-propanol, pentane, 1-pentanol, 1-propanol, 2-propanol, propylacetate, triethylamine, 1,1-diethoxypropane, 1,1-dimethoxymethane, 2,2-dimethoxypropane, isooctane, isopropyl ether, methylisopropyl ketone, methyltetrahydrofuran, petroleum ether, trichloroacetic acid, trifluoroacetic acid, decanol, 2-ethylhexylacetate, amylacetate, or a combination thereof.

17. The method of any one of claims 2-16, wherein step ii. further comprises adding one or more different excipients to each container.

18. The method of any one of the preceding claims, wherein the one or more different excipients is selected from one or more of a carbohydrate, a salt, a chelator, a mineral, a polymer, a surfactant, a protein stabilizer, an emulsifier, an antiseptic, an amino acid, an antioxidant, an organic solvent, a paraben, a bactericide, a preservative, and an analgesic.

19. The method of claim 18, wherein the carbohydrate is dextran, trehalose, sucrose, agarose, mannitol, lactose, sorbitol, maltose, or a combination thereof.

20. The method of claim 18, wherein the salt is sodium chloride, calcium chloride, potassium chloride, sodium hydroxide, stannous chloride, magnesium sulfate, sodium glucoheptonate, sodium pertechnetate, guanidine hydrochloride, potassium hydroxide, magnesium chloride, potassium nitrate, or a combination thereof.

21. The method of claim 18, wherein the chelator is disodium edetate, ethylenediaminetetraacetic acid or pentetic acid.

22. The method of claim 18, wherein the mineral is calcium, zinc, or titanium dioxide.

23. The method of claim 18, wherein the polymer is propyleneglycol, glucose star polymer, silicone polymer, polydimethylsiloxane, polyethylene glycol, carboxymethylcellulose, poly(glycolic acid), poly(lactic-co-glycolic acid), polylactic acid, polycaprolactone (PCL), polyvinylpyrrolidone (PVP), ficoll, dextran, or a combination thereof.

24. The method of claim 18, wherein the surfactant is polysorbate, magnesium stearate, sodium dodecyl sulfate, alkylphenol ethoxylates, glycerin, polyoxyethylated castor oil, docusate, sodium stearate, decyl glucoside, nonoxynol-9, cetyltrimethylammonium bromide, sodium bis(2-ethylhexyl) sulfosuccinate, lecithin, sorbitan esters, or a combination thereof.

25. The method of claim 18, wherein the protein stabilizer is trehalose, polyethylene glycol (PEG), polyoxamers, polyvinylpyrrolidone, polyacrylic acids, poly(vinyl) polymers, polyesters, polyaldehydes, tert-polymers, polyamino acids, hydroxyethylstarch, N-methyl-2-pyrrolidone, sorbitol, sucrose, mannitol, cyclodextrin, hydroxypropyl beta-cyclodextrin, sulfobutylether beta-cyclodextrin, or a combination thereof.

26. The method of claim 18, wherein the emulsifier is polysorbate, sorbitan monooleate, ethanolamine, polyoxyl 35 castor oil, poloxyl 40 hydrogenated castor oil, carbomer 1342, a corn oil-mono-di-triglyceride, a polyoxyethylated oleic glyceride, a poloxamer, or a combination thereof.

27. The method of claim 18, wherein the antiseptic is phenol, m-cresol, benzyl alcohol, 2-phenyloxyethanol, chlorobutanol, neomycin, benzethonium chloride, gluteraldehyde, beta-propiolactone, or a combination thereof.

28. The method of claim 18, wherein the amino acid is aspartic acid, cysteine, isoleucine, glutamic acid, leucine, methionine, phenylalanine, pyrrolysine, serine, selenocysteine, threonine, tryptophan, tyrosine, valine, asparagine, arginine, histidine, glutamine, proline, or a combination thereof.

29. The method of claim 18, wherein the antioxidant is glutathione, ascorbic acid, cysteine, N-acetyl-L-tryptophanate, tocopherol, histidine, methionine, or a combination thereof.

30. The method of claim 18, wherein the organic solvent is dimethyl sulfoxide, N-methyl-2-pyrrolidone, or a combination thereof.

31. The method of claim 18, wherein the paraben is a parahydroxybenzoate.

32. The method of claim 18, wherein the bactericide is benzalkonium chloride or benzyl benzoate.

33. The method of claim 18, wherein the preservative is methyl hydroxybenzoate, thimerosal, a paraben, formaldehyde, castor oil, or a combination thereof.

34. The method of claim 18, wherein the analgesic is acetaminophen or lidocaine.

35. The method of any one of the preceding claims, wherein the excipient further comprises at least one pharmaceutically acceptable additive, diluent, carrier, or a combination thereof.

36. The method of any one of claims 2-35, wherein step iv. comprises mixing the first aqueous liquid comprising the protein, pH adjusting agent and one or more excipients with the organic liquid, thereby forming particles comprising the protein, pH adjusting agent and one or more excipients in each container, wherein the circularity of the particles in each well is from about 0.80 to about 1.00.

37. The method of any one of the preceding claims, wherein the mixing comprises mechanical stirring, mechanical shaking, mechanical stirring, vortexing, or sonication.

38. The method of any one of the preceding claims, wherein the mixing comprises vortexing or sonication.

39. The method of any one of the preceding claims, wherein the particles have a circularity of about 0.85 to about 1.00.

40. The method of any one of the preceding claims, wherein the particles have a circularity of about 0.90 to about 1.00.

41. The method of any one of the preceding claims, wherein the particles have a circularity of about 0.95 to about 1.00.

42. The method of any one of the preceding claims, wherein the particles have a circularity of about 0.98 to about 1.00.

43. The method of any one of the preceding claims, wherein the particles have a circularity of about 1.00.

44. The method of any one of the preceding claims, wherein the particles have less than about 10% internal void spaces.

45. The method of any one of the preceding claims, wherein the particles have less than about 5% internal void spaces.

46. The method of any one of the preceding claims, wherein the particles in step d) have less than about 3% internal void spaces.

47. The method of any one of the preceding claims, wherein the particles have less than about 1% internal void spaces.

48. The method of any one of the preceding claims, wherein the particles are substantially free from any internal void spaces.

49. The method of any one of the preceding claims, wherein the particles have greater than about 60% protein by weight.

50. The method of any one of the preceding claims, wherein the particles have greater than about 70% protein by weight.

51. The method of any one of the preceding claims, wherein the particles have greater than about 80% protein by weight.

52. The method of any one of the preceding claims, wherein the particles have greater than about 90% protein by weight.

53. The method of any one of the preceding claims, wherein the particles have greater than about 95% protein by weight.

54. The method of any one of the preceding claims, wherein the particles have greater than about 98% protein by weight.

55. The method of any one of the preceding claims, wherein the dispensing in step a) is performed manually or by automated liquid handling.

56. The method of any one of the preceding claims, wherein the first aqueous liquid and organic liquid in step b) is removed by lyophilization or vacuum desiccation.

57. The method of any one of the preceding claims, wherein the stability of the protein in step c) is determined by measuring aggregation, fragmentation, change in charge variants, Subvisible Particles (SvPs), turbidity, or a combination thereof, of the protein.

58. The method of any one of the preceding claims, wherein the particles in step d) have less than about 5% aggregation of the protein.

59. The method of any one of the preceding claims, wherein the particles in step d) have less than about 3% aggregation of the protein.

60. The method of any one of the preceding claims, wherein the particles in step d) have less than about 1% aggregation of the protein.

61. The method of any one of the preceding claims, wherein the particles in step d) have less than about 0.5% aggregation of the protein.

62. The method of any one of the preceding claims, wherein the particles in step d) are substantially free from any aggregation of the protein.

63. The method of any one of the preceding claims, wherein the particles in step d) have less than about 5% fragmentation of the protein.

64. The method of any one of the preceding claims, wherein the particles in step d) have less than about 3% fragmentation of the protein.

65. The method of any one of the preceding claims, wherein the particles in step d) have less than about 1% fragmentation of the protein.

66. The method of any one of the preceding claims, wherein the particles in step d) are substantially free from any fragmentation of the protein.

67. The method of any one of the preceding claims, wherein the particles in step d) have less than about 5% change in charge variants of the protein.

68. The method of any one of the preceding claims, wherein the particles in step d) have less than about 3% change in charge variants of the protein.

69. The method of any one of the preceding claims, wherein the particles in step d) have less than about 1% change in charge variants of the protein.

70. The method of any one of the preceding claims, wherein the particles in step d) are substantially free from any change in charge variants of the protein.

71. The method of any one of the preceding claims, wherein the particles in step d) are substantially free of Visible Particles (VP) upon dissolution in a second aqueous liquid.

72. The method of any one of the preceding claims, wherein the particles in step d) have a concentration of insoluble Subvisible Particles (SvPs) having a characteristic size of greater than about 10 μm of about 0 per mL to about 100,000,000 per mL upon dissolution in a second aqueous liquid.

73. The method of any one of the preceding claims, wherein the particles in step d) have a concentration of insoluble Subvisible Particles (SvPs) having a characteristic size of greater than about 10 μm of about 0 per mL to about 6000 per mL upon dissolution in a second aqueous liquid.

74. The method of any one of the preceding claims, wherein the particles in step d) have a concentration of insoluble Subvisible Particles (SvPs) having a characteristic size of greater than about 25 μm of about 0 per mL to about 600 per mL upon dissolution in a second aqueous liquid.

75. The method of any one of the preceding claims, wherein the particles in step d) are substantially free of insoluble Subvisible Particles (SvPs) upon dissolution in a second aqueous liquid.

76. The method of any one of the preceding claims, wherein the particles in step d) upon dissolution in a second aqueous liquid have a substantially similar turbidity compared to a composition comprising the protein in the first aqueous liquid.

77. The method of any one of the preceding claims, wherein the particles in step d) upon dissolution in a second aqueous liquid are substantially free of turbidity.

78. The method of any one of the preceding claims, wherein the second aqueous liquid is water, aqueous buffer or a physiologically relevant aqueous liquid.

79. The method of claim 78, wherein the aqueous buffer is phosphate buffer, or phosphate-buffered saline (PBS).

80. The method of any one of the preceding claims, wherein the particles in step d) have improved stability of the protein compared to a composition comprising the protein in the first aqueous liquid.

81. The method of any one of the preceding claims, wherein the particles in step d) are stable for at least one month.

82. The method of any one of the preceding claims, wherein the particles in step d) are stable for at least two months.

83. The method of any one of the preceding claims, wherein the particles in step d) are stable for at least three months.

84. The method of claim 81, wherein the particles in step d) are stable for at least one month at 60° C.

85. The method of claim 83, wherein the particles in step d) are stable for at least three months at 40° C.

86. The method of claim 83, wherein the particles in step d) are stable for at 12 months at 40° C.

87. The method of any one of the preceding claims, wherein the method is automated.
